SEQUENCE LISTING (76 sequences)

SEQ ID NO: 1 - DNA, 300 bp, Methylobacterium sp.
  source 1..300: mol_type = genomic DNA
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 2 - DNA, 300 bp, Methylobacterium sp.
  source 1..300: mol_type = genomic DNA
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 3 - DNA, 300 bp, Methylobacterium sp.
  source 1..300: mol_type = genomic DNA
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 4 - DNA, 23 bp, synthetic construct
  misc_feature 1..23: note = Synthetic
  source 1..23: mol_type = other DNA
cctcaccggtgttctatcataac

SEQ ID NO: 5 - DNA, 20 bp, synthetic construct
  misc_feature 1..20: note = Synthetic
  source 1..20: mol_type = other DNA
ccgatgatggtggtctgaac

SEQ ID NO: 6 - DNA, 21 bp, synthetic construct
  misc_feature 1..21: note = Synthetic
  source 1..21: mol_type = other DNA
cgtcccttatgcgatgtacca

SEQ ID NO: 7 - DNA, 22 bp, synthetic construct
  misc_feature 1..22: note = Synthetic
  source 1..22: mol_type = other DNA
gatccgagcctaagactcaaag

SEQ ID NO: 8 - DNA, 21 bp, synthetic construct
  misc_feature 1..21: note = Synthetic
  source 1..21: mol_type = other DNA
gaccaatgcctatccgatcaa

SEQ ID NO: 9 - DNA, 20 bp, synthetic construct
  misc_feature 1..20: note = Synthetic
  source 1..20: mol_type = other DNA
aacacttggtagagacagcc

SEQ ID NO: 10 - DNA, 21 bp, synthetic construct
  misc_feature 1..21: note = Synthetic
  source 1..21: mol_type = other DNA
aaggagggatggaagcgaaac

SEQ ID NO: 11 - DNA, 19 bp, synthetic construct
  misc_feature 1..19: note = Synthetic
  source 1..19: mol_type = other DNA
ataacccacgcttgccttc

SEQ ID NO: 12 - DNA, 21 bp, synthetic construct
  misc_feature 1..21: note = Synthetic
  source 1..21: mol_type = other DNA
cgcagagtaaccgccgacgaa

SEQ ID NO: 13 - DNA, 300 bp, Methylobacterium sp.
  source 1..300: mol_type = genomic DNA
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 14 - DNA, 300 bp, Methylobacterium sp.
  source 1..300: mol_type = genomic DNA
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 15 - DNA, 300 bp, Methylobacterium sp.
  source 1..300: mol_type = genomic DNA
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 16 - DNA, 21 bp, synthetic construct
  misc_feature 1..21: note = Synthetic
  source 1..21: mol_type = other DNA
gtcctccgaaacccacataaa

SEQ ID NO: 17 - DNA, 23 bp, synthetic construct
  misc_feature 1..23: note = Synthetic
  source 1..23: mol_type = other DNA
ctaccagaagcaatgaaagtcat

SEQ ID NO: 18 - DNA, 22 bp, synthetic construct
  misc_feature 1..22: note = Synthetic
  source 1..22: mol_type = other DNA
tctgtaatagcctgccaaagca

SEQ ID NO: 19 - DNA, 19 bp, synthetic construct
  misc_feature 1..19: note = Synthetic
  source 1..19: mol_type = other DNA
ggctggtgctcaaggtaat

SEQ ID NO: 20 - DNA, 20 bp, synthetic construct
  misc_feature 1..20: note = Synthetic
  source 1..20: mol_type = other DNA
acatttagggcggtcaagag

SEQ ID NO: 21 - DNA, 19 bp, synthetic construct
  misc_feature 1..19: note = Synthetic
  source 1..19: mol_type = other DNA
atgaagtgatccgggccat

SEQ ID NO: 22 - DNA, 21 bp, synthetic construct
  misc_feature 1..21: note = Synthetic
  source 1..21: mol_type = other DNA
ccgtactgcatagggatgaaa

SEQ ID NO: 23 - DNA, 21 bp, synthetic construct
  misc_feature 1..21: note = Synthetic
  source 1..21: mol_type = other DNA
taaggcaaaggatgggatcaa

SEQ ID NO: 24 - DNA, 21 bp, synthetic construct
  misc_feature 1..21: note = Synthetic
  source 1..21: mol_type = other DNA
ttgctgacgaggttgtggtag

SEQ ID NO: 25 - DNA, 300 bp, Methylobacterium sp.
  source 1..300: mol_type = genomic DNA
caactatgtagacccgacggtgcgatttcacttcgcaaagccgcagggcagcacccttgcgctcaatgttgacgccagcgtgatctatactattaccgtcacgcacacgcagggcggcgtacagattcatcgcgagagtaagaaccaccatcagaccatcacgcgcagcgacctgagcaagcagttcggcgttggtgtggccgaccagctgacgcgcgatcaggtcatgaaggtgatcgagtcggcatttcgcgacgctacccgctaagatcggcgcccacgaaacgctacgagactagg

SEQ ID NO: 26 - DNA, 300 bp, Methylobacterium sp.
  source 1..300: mol_type = genomic DNA
agccggcatcttgttcaaggcgctcacctcgacgccgacgctgtaggcgacttgagagggcgtctcatatgaacgaagcatcttcgcgtagagaaccttcttgttctcctgcgtgatgttcgctttgcagacgttgactgccgccatgaacgccgaagccttgcgcgcttcatcgtaatcgcctgcgaaggcgggtagtgaaaagcttagtgcaatggcaaacacagccgccgaacgtcgcatggtatccgtccccgattgacggcagtgccgccatatctcggctttagcagagctgat

SEQ ID NO: 27 - DNA, 300 bp, Methylobacterium sp.
  source 1..300: mol_type = genomic DNA
aacctgcgccggccgaggtttcgcgagccgtcgccacgggcaacgcctcgcccgcgatgtgcaaaaaagtccccggcacttcgcgccgtcgtccgatccacgaccgcgaatttctcaacgagtacaaggtgcttatgggagatccgagcgtccgtcccggagcccgagaccgcgcggcccgagtaataggcgaaaaagactcctactcctcgggcttctcgggccccctcagcaacatctacgcttgccgcccatcaccctggcgggagatcagcgacgagacacaggcccacttcgccc

SEQ ID NO: 28 - DNA, 22 bp, synthetic construct
  misc_feature 1..22: note = Synthetic
  source 1..22: mol_type = other DNA
ttgacgccagcgtgatctatac

SEQ ID NO: 29 - DNA, 22 bp, synthetic construct
  misc_feature 1..22: note = Synthetic
  source 1..22: mol_type = other DNA
gtgatggtctgatggtggttct

SEQ ID NO: 30 - DNA, 19 bp, synthetic construct
  misc_feature 1..19: note = Synthetic
  source 1..19: mol_type = other DNA
tattaccgtcacgcacacg

SEQ ID NO: 31 - DNA, 24 bp, synthetic construct
  misc_feature 1..24: note = Synthetic
  source 1..24: mol_type = other DNA
cttcgcgtagagaaccttcttgtt

SEQ ID NO: 32 - DNA, 22 bp, synthetic construct
  misc_feature 1..22: note = Synthetic
  source 1..22: mol_type = other DNA
cttcgcaggcgattacgatgaa

SEQ ID NO: 33 - DNA, 20 bp, synthetic construct
  misc_feature 1..20: note = Synthetic
  source 1..20: mol_type = other DNA
cgtgatgttcgctttgcaga

SEQ ID NO: 34 - DNA, 23 bp, synthetic construct
  misc_feature 1..23: note = Synthetic
  source 1..23: mol_type = other DNA
ccgcgaatttctcaacgagtaca

SEQ ID NO: 35 - DNA, 21 bp, synthetic construct
  misc_feature 1..21: note = Synthetic
  source 1..21: mol_type = other DNA
gcccgaggagtaggagtcttt

SEQ ID NO: 36 - DNA, 20 bp, synthetic construct
  misc_feature 1..20: note = Synthetic
  source 1..20: mol_type = other DNA
aggtgcttatgggagatccg

SEQ ID NO: 37 - DNA, 300 bp, Methylobacterium sp.
  source 1..300: mol_type = genomic DNA
agtcattgatcaagcaacccctattgagttggatatcgaaggatcaaggtcgcgtcaatagatgcatctatcaggccaaatgtcgcttttcaagaatggctctttcgaagctatctttataatcgctcgccattctctcattaccaaaatcgaccttaactagctcgacattgatgcgagcagctccggcaaacgaggagagattgaccttaaaggaattgaacgcctcaagcaattcagacacattaccaggagtgctatagcaacaaccagacccatatcggtcaataacctctttta

SEQ ID NO: 38 - DNA, 300 bp, Methylobacterium sp.
  source 1..300: mol_type = genomic DNA
cgcaaaacgatttatcactgccatcttgttgtttgataacccttttttaccagacgttatgctgggcgagaaagaggactagcagatcggagcggtatcgcgatttttcggtagttcgcgcctacaacaggataagatccgatagtgaagcaacatggctgttttttgatttgtaagtcagcaacttaagcagccagcctatctgccgtcgcagacgcttgaggcatcgggcagcatcttagaaaaggtggcagtaattgccacagcggaacgtagcggcacggataagcacgcagggtc

SEQ ID NO: 39 - DNA, 300 bp, Methylobacterium sp.
  source 1..300: mol_type = genomic DNA
cccatctggacccaatatccccttcatcgacaattcccgagtaagtgtgggttcgaggatttcgcgaaacagccttgttcgttcctccggccttaaaattggcgtgccgtcgggagatcgataggcatcccttacctgcctttcgaccgccggcacacgcgcgccggtcgtcgtgttcacggccacggaatggacgaaggtgcgccgctcatttcgctcgtttgccgtctccaccatccaggaggccagcaggacggtttcgtctcgaccgccggtcacacacaccgcaagggactcagg

SEQ ID NO: 40 - DNA, 22 bp, synthetic construct
  misc_feature 1..22: note = Synthetic
  source 1..22: mol_type = other DNA
tcgctcgccattctctcattac

SEQ ID NO: 41 - DNA, 22 bp, synthetic construct
  misc_feature 1..22: note = Synthetic
  source 1..22: mol_type = other DNA
aggtcaatctctcctcgtttgc

SEQ ID NO: 42 - DNA, 18 bp, synthetic construct
  misc_feature 1..18: note = Synthetic
  source 1..18: mol_type = other DNA
tcgacattgatgcgagca

SEQ ID NO: 43 - DNA, 22 bp, synthetic construct
  misc_feature 1..22: note = Synthetic
  source 1..22: mol_type = other DNA
ttcgcgcctacaacaggataag

SEQ ID NO: 44 - DNA, 23 bp, synthetic construct
  misc_feature 1..23: note = Synthetic
  source 1..23: mol_type = other DNA
cagataggctggctgcttaagtt

SEQ ID NO: 45 - DNA, 18 bp, synthetic construct
  misc_feature 1..18: note = Synthetic
  source 1..18: mol_type = other DNA
tccgatagtgaagcaaca

SEQ ID NO: 46 - DNA, 24 bp, synthetic construct
  misc_feature 1..24: note = Synthetic
  source 1..24: mol_type = other DNA
gagtaagtgtgggttcgaggattt

SEQ ID NO: 47 - DNA, 23 bp, synthetic construct
  misc_feature 1..23: note = Synthetic
  source 1..23: mol_type = other DNA
aggtaagggatgcctatcgatct

SEQ ID NO: 48 - DNA, 18 bp, synthetic construct
  misc_feature 1..18: note = Synthetic
  source 1..18: mol_type = other DNA
cggaggaacgaacaaggc

SEQ ID NO: 49 - DNA, 300 bp, Methylobacterium sp.
  source 1..300: mol_type = genomic DNA
acctgctaaaatcacgtcctctcagattgaaaaatcattgaagaaacgtgtcgaacgattgccggggattatgacgttagatcaattgaaaaatacaagctttgaaattgagttacagccaaaagatgccccggatccggacccatcagacttcggtggctagttcgagccaaactcgaacgtcgccatggcgcgcaagtcgcaataccatttcacagcgcagcggttatttcgttgtacactgtagcaatgcgtcggcttgcgcgcttccgctggcgatcaaaggtccgccgatttacg

SEQ ID NO: 50 - DNA, 300 bp, Methylobacterium sp.
  source 1..300: mol_type = genomic DNA
tcccgaacatacaatggaggaagcgtgtggtaggccaatttgtaacgaaatatggcatcggtcacggctctctcaataaattcgatctcaagtcttctgaacgagcatgcctcatccttatcctgagcgaacgcctgccagtttgcagtcattccaacatacatagccaaaaaggcgaggtagaccttcatacgggcacctcaatcgtccccattcgttcaagctccttcaagataacagccgcaccacattgctgagatcgaagattcggatcaaatattccatcaaatttatactttc

SEQ ID NO: 51 - DNA, 300 bp, Methylobacterium sp.
  source 1..300: mol_type = genomic DNA
gcatcctttgcgctcgcaggcctaaggtcaagcccggttacttcgtttggtagaacgaggtagacgatgcctagtcttaaggtggcccatgttaaccaacagggccagaacatgattatagttccgttagatgccaacttcggttacaaaaccgatggtgagcagtccgacatcatgttcgaaatacaggacgcggcgcggtccgccggtcttgcgggtgccgtagtagcgttctggcagtcaggtggacaaacccgtttccggggcccggctccgtggcacccattccttcgcagcctc

SEQ ID NO: 52 - DNA, 22 bp, synthetic construct
  misc_feature 1..22: note = Synthetic
  source 1..22: mol_type = other DNA
gcgcaagtcgcaataccatttc

SEQ ID NO: 53 - DNA, 21 bp, synthetic construct
  misc_feature 1..21: note = Synthetic
  source 1..21: mol_type = other DNA
cgtaaatcggcggacctttga

SEQ ID NO: 54 - DNA, 19 bp, synthetic construct
  misc_feature 1..19: note = Synthetic
  source 1..19: mol_type = other DNA
cgcagcggttatttcgttg

SEQ ID NO: 55 - DNA, 22 bp, synthetic construct
  misc_feature 1..22: note = Synthetic
  source 1..22: mol_type = other DNA
acgagcatgcctcatccttatc

SEQ ID NO: 56 - DNA, 21 bp, synthetic construct
  misc_feature 1..21: note = Synthetic
  source 1..21: mol_type = other DNA
cgattgaggtgcccgtatgaa

SEQ ID NO: 57 - DNA, 20 bp, synthetic construct
  misc_feature 1..20: note = Synthetic
  source 1..20: mol_type = other DNA
tgccagtttgcagtcattcc

SEQ ID NO: 58 - DNA, 23 bp, synthetic construct
  misc_feature 1..23: note = Synthetic
  source 1..23: mol_type = other DNA
cccggttacttcgtttggtagaa

SEQ ID NO: 59 - DNA, 24 bp, synthetic construct
  misc_feature 1..24: note = Synthetic
  source 1..24: mol_type = other DNA
cgaagttggcatctaacggaacta

SEQ ID NO: 60 - DNA, 20 bp, synthetic construct
  misc_feature 1..20: note = Synthetic
  source 1..20: mol_type = other DNA
tggcccatgttaaccaacag

SEQ ID NO: 61 - DNA, 300 bp, Methylobacterium sp.
  source 1..300: mol_type = genomic DNA
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 62 - DNA, 300 bp, Methylobacterium sp.
  source 1..300: mol_type = genomic DNA
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 63 - DNA, 300 bp, Methylobacterium sp.
  source 1..300: mol_type = genomic DNA
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 64 - DNA, 300 bp, Methylobacterium sp.
  source 1..300: mol_type = genomic DNA
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 65 - DNA, 300 bp, Methylobacterium sp.
  source 1..300: mol_type = genomic DNA
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 66 - DNA, 300 bp, Methylobacterium sp.
  source 1..300: mol_type = genomic DNA
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 67 - DNA, 300 bp, Methylobacterium sp.
  source 1..300: mol_type = genomic DNA
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 68 - DNA, 300 bp, Methylobacterium sp.
  source 1..300: mol_type = genomic DNA
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 69 - DNA, 300 bp, Methylobacterium sp.
  source 1..300: mol_type = genomic DNA
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 70 - DNA, 300 bp, Methylobacterium sp.
  source 1..300: mol_type = genomic DNA
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 71 - DNA, 300 bp, Methylobacterium sp.
  source 1..300: mol_type = genomic DNA
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 72 - DNA, 300 bp, Methylobacterium sp.
  source 1..300: mol_type = genomic DNA
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 73 - DNA, 299 bp, Methylobacterium sp.
  source 1..299: mol_type = genomic DNA
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

SEQ ID NO: 74 - DNA, 300 bp, Methylobacterium sp.
  source 1..300: mol_type = genomic DNA
accgaaggcgtccccggacacgaaggcctgaaacaccatatctgtggcgatcaggccgacgtggtcgcggacttcaactggcagagaatgccaggccgcttcgatttcagatgatactggtacggacataggagcggcttagctttctcagtgcaaatgtgattgattccggctcaaaaatgatcttgatcggacgagacgttttcaatccatgtcgtgttgccatcgccgatcggtgcgtcaagagacagatggcgccgaccgtagatacgcgttcgggttgcccgcaccgcttctcca

SEQ ID NO: 75 - DNA, 300 bp, Methylobacterium sp.
  source 1..300: mol_type = genomic DNA
ggaggtgtgatctgatgatgtgctggatgaaattggcggtcgagcacttgttcagcttggccagctcgacgagatcggcgtgatgctcggcgtcgatcaggatgttcagcgagaccggacgtacgcaggacttggtattagcgccgttgcgcatcagcttgcagccttgctctgcttctcagcgtgccgcgtcaggatgaccctgatgtagctgttgaggttgatgccgtaatagcctgcggactctgtgagatcccggcgaagatcgtcggcgagggtcaggcggatggtgctggtcgg

SEQ ID NO: 76 - DNA, 300 bp, Methylobacterium sp.
  source 1..300: mol_type = genomic DNA
aagtaaccgctcaacatgatcttcagcatgttgtccaacagcaggagaatacatgtaattcaccatgaccggcaagctgcgactggccattgcttccaccgcttgaatgtagcgatcgaatttcgcaaaatcagggtggaatgaaaatatcgaaccaaactgcgagccttgaatccgttctgcaaaattatcgaaaaattttcttggccgactgccgttcgaaaacattcttacgtttacatgcggcccgcctgaaacaagacagtctaccagctctgggaaatgggggtgaagggtcgg

(19) United States
(12) Patent Application Publication
(10) Pub. No.: US 2025/0241314 A1
(43) Pub. Date: Jul. 31, 2025

(54) METHODS FOR OBTAINING AND USING PLANTS AND PLANT PARTS WITH INCREASED NUTRIENT, OIL, AND/OR PROTEIN CONTENT

(71) Applicant: NewLeaf Symbiotics, Inc., St. Louis, MO (US)

(72) Inventors: Patrick Vogan, Shrewsbury, MO (US); Janne Kerovuo, Los Angeles, CA (US); Charles Michael McFatrich, Washington, MO (US); Natalie Breakfield, St. Louis, MO (US)

(21) Appl. No.: 19/029,217

(22) Filed: Jan. 17, 2025

Related U.S. Application Data
(62) Division of application No. 17/309,505, filed on Jun. 2, 2021, now Pat. No. 12,245,594.
(60) Provisional application No. 62/900,766, filed on Sep. 16, 2019.
(60) Provisional application No. 62/878,164, filed on Jul. 24, 2019.
(60) Provisional application No. 62/846,247, filed on May 10, 2019.
(60) Provisional application No. 62/802,038, filed on Feb. 6, 2019.
(60) Provisional application No. 62/774,640, filed on Dec. 3, 2018.

(51) Int. Cl. A01N 63/20; A23K 10/30; A23K 20/147; A23K 20/158; A23K 20/174; A23K 20/20; A23K 20/22; A23K 20/24; A23K 20/26; A23K 50/10; A23K 50/30; A23K 50/75; A23K 50/80; A23L 33/105; A23L 33/115; A23L 33/15; A23L 33/16; A23L 33/185; C12Q 1/689

(52) U.S. Cl. CPC A01N 63/20; A23K 10/30; A23K 20/147; A23K 20/158; A23K 20/174; A23K 20/22; A23K 20/24; A23K 20/26; A23K 20/30; A23K 50/10; A23K 50/30; A23K 50/75; A23K 50/80; A23L 33/105; A23L 33/115; A23L 33/15; A23L 33/16; A23L 33/185; C12Q 1/689

(57) ABSTRACT

Methods for identifying Methylobacterium strains which can be used to increase mineral nutrient, vitamins, oil and/or protein content in plants are provided. Also provided are related methods of providing seed lots, food ingredients, feed ingredients, food, or feed with increased mineral nutrient, vitamins, oil and/or protein content. Methods of providing increased mineral nutrient and/or vitamin content, and/or increased oil and/or protein yield from plant seed lots are also provided.

### CROSS-REFERENCE TO RELATED APPLICATIONS

This patent application is a divisional application of U.S. Ser. No. 17/309,505, filed Jun. 2, 2021, which is a U.S. national phase application claiming priority to PCT/US2019/064033, filed Dec. 2, 2019, which claims the benefit of U.S. 62/774,640, filed Dec. 3, 2018, U.S. 62/802,038, filed Feb. 6, 2019, U.S. 62/846,247, filed May 10, 2019, U.S. 62/878,164, filed Jul. 24, 2019, and U.S. 62/900,766, filed Sep. 16, 2019, which are each incorporated herein by reference in their entireties.

### SEQUENCE LISTING STATEMENT

A sequence listing containing the XML file named “P13523US06_SequenceListing.xml” which is 93,609 bytes (measured in MS-Windows®) and created on Jan. 15, 2025, contains 76 nucleotide sequences, is provided herewith via the USPTO's EFS system, and is incorporated herein by reference in its entirety.

### BACKGROUND

Plant seeds are an important source of oil for use in food, feed, and other industrial applications. Such oil can be obtained from plant seeds by a variety of methods including expelling and/or solvent extraction. Provision of seeds with increased oil content is anticipated to provide for improved oil yield in oil production processes where plant seed are used as feedstock. Provision of seeds with increased oil content is also anticipated to provide seeds that can be used in either a whole or processed form to provide food ingredients, feed ingredients, food, or feed with increased oil content.

Plant seeds are also an important source of protein for use in food, feed, and other industrial applications. Such protein can be obtained from plant seeds by a variety of methods including extraction of defatted seed meal with water or other aqueous solvents. Provision of seeds with increased protein content is anticipated to provide for improved protein yield production processes where plant seeds are used as feedstock. Provision of seeds with increased protein content is also anticipated to provide seeds that can be used in either a whole or processed form to provide food ingredients, feed ingredients, food, or feed with increased protein content.

Plants require certain macronutrients and micronutrients for growth and metabolism. These elements are generally found in the soil as salts and can be consumed by plants as ions. In agriculture, soil can become depleted of one or more of these nutrients requiring the addition of fertilizers to provide sufficient quantities of the nutrients for crop growth. Plants with such macronutrients and micronutrients can also be a source of such nutrients in human and animal diets. Thus, methods of increasing levels of macronutrients and micronutrients in plants are desired for benefits to agricultural practices and to human and animal nutrition.

One-carbon organic compounds such as methane and methanol are found extensively in nature, and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase which incorporates an atom of oxygen from O₂ into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Some methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M. chloromethanicum, M. dichloromethanicum, M. extorquens, M. fujisawaense, M. mesophilicum, M. organophilum, M. radiotolerans, M. rhodesianum, M. rhodinum, M. thiocyanatum, and M. zatmanii. However, M. nodulans is a nitrogen-fixing Methylobacterium that is not a PPFM (Sy et al., 2001). Methylobacterium are found in soil, dust, fresh water, sediments, and leaf surfaces, as well as in industrial and clinical environments (Green, 2006).

Methylobacterium strains that can be applied to a variety of crops including corn and soybean to improve seed yield have been reported in US Patent Application Publication Nos. 20160295868 and 20160302423, respectively.

### SUMMARY

Methods for identifying a Methylobacterium strain that increases mineral nutrient, vitamin, crude fat, seed oil, and/or protein content comprising: (i) treating a seed and/or a plant with at least a first Methylobacterium strain to obtain a treated seed and/or a treated plant; (ii) harvesting progeny seed from a mature treated plant, wherein the mature treated plant is grown from the treated seed or treated plant of step (i); (ii) harvesting progeny seed from a mature control plant wherein the mature control plant was grown from an untreated control seed or untreated control plant; (iii) determining mineral nutrient, vitamin, oil and/or protein content in the progeny seed from the mature treated plant and from the mature control plant; and, (iv) selecting a Methylobacterium strain that increases the content of one or more mineral nutrients and/or vitamins, and/or the content of crude fat, seed oil, and/or protein of the progeny seed from the mature treated plant in comparison to the mineral nutrient, vitamin, crude fat, seed oil, and/or protein from the progeny seed from the mature control plant are provided.

Methods of identifying a Methylobacterium strain that increases mineral nutrient, vitamin, crude fat, seed oil, and/or protein content comprising: (i) treating a first seed or plant part with at least a first Methylobacterium strain and a second seed or plant part with a second Methylobacterium strain, (ii) planting a control seed or plant part, the first seed or plant part, and the second seed or plant part, wherein the control seed or plant part is not treated with the first Methylobacterium strain or the second Methylobacterium strain; (iii) harvesting one or more progeny seed from a plant grown from the first seed or plant part, from the plant grown from the second seed or plant part, and from the plant grown from the control seed or plant part; (iv) analyzing the progeny seed harvested from the plant grown from the first seed or plant part, from the plant grown from the second seed or plant part, and from the plant grown from the control seed or plant part for mineral nutrient, vitamin, oil and/or protein content; and (v) selecting the Methylobacterium strain that provides progeny seed with the greatest increases in mineral nutrient, vitamin, oil and/or protein content in comparison to progeny seed from the plant grown from the control seed, wherein the Methylobacterium strain is either the first Methylobacterium strain or the second Methylobacterium strain are provided.

Methods of producing a food or feed ingredient with increased mineral nutrient, vitamin, crude fat, oil and/or protein content comprising dehulling, delinting, crushing, macerating, grinding, and/or extracting a seed lot wherein at least 50%, 70%, 80%, 90%, or 95% of the seeds in the seed lot were harvested from mature plants grown from seeds and/or plants treated with an effective amount of a Methylobacterium strain, thereby obtaining a food or feed ingredient with increased oil and/or protein content are provided. In certain embodiments of such methods, the Methylobacterium is ISO10 (NRRL B-50938) or ISO20 (NRRL B-67743). In certain embodiments, the Methylobacterium has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

Methods of producing a food or feed with increased mineral nutrient, vitamin, crude fat, oil and/or protein content comprising incorporating into the food or feed a processed or unprocessed food ingredient obtained from a seed lot wherein at least 50%, 70%, 80%, 90%, or 95% of the seeds in the seed lot were harvested from mature plants grown from seeds and/or plants treated with an effective amount of a Methylobacterium strain, thereby obtaining a food or feed with increased mineral nutrient, vitamin, oil and/or protein content are provided. In certain embodiments of such methods, the Methylobacterium strain is ISO10 (NRRL B-50938) or ISO20 (NRRL B-67743). In certain embodiments, the Methylobacterium strain has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium strain has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

Methods of improving seed oil yield from a seed lot comprising separating an oil-enriched fraction from a seed lot wherein at least 50%, 70%, 80%, 90%, or 95% of the seeds in the seed lot were harvested from mature plants grown from seeds and/or plants treated with an effective amount of a Methylobacterium strain are provided. In certain embodiments of such methods, the Methylobacterium strain is ISO10 (NRRL B-50938) or ISO20 (NRRL B-67743). In certain embodiments, the Methylobacterium strain has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium strain has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

Methods of improving seed protein yield from a seed lot comprising: (i) obtaining a seed lot wherein at least 50%, 70%, 80%, 90%, or 95% of the seed in the seed lot were harvested from mature plants grown from seeds and/or plants treated with an effective amount of a Methylobacterium strain; and (ii) separating a protein-enriched fraction from the seed lot are provided. In certain embodiments of such methods, the Methylobacterium strain is ISO10 (NRRL B-50938) or ISO20 (NRRL B-67743). In certain embodiments, the Methylobacterium strain has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium strain has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

Methods of improving seed protein yield from a seed lot comprising separating a protein-enriched fraction from a seed lot wherein at least 50%, 70%, 80%, 90%, or 95% of the seed in the seed lot were harvested from mature plants grown from seeds and/or plants treated with an effective amount of a Methylobacterium strain are provided. In certain embodiments of such methods, the Methylobacterium strain is ISO10 (NRRL B-50938) or ISO20 (NRRL B-67743). In certain embodiments, the Methylobacterium strain has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium strain has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

Methods of providing a seed lot with increased crude fat, oil, and/or protein content comprising harvesting a seed lot from mature plants grown from seeds and/or plants treated with an effective amount of a Methylobacterium strain, wherein at least 95% of the seeds in the harvested seed lot are obtained from the mature plants, wherein the harvested seed lot is packaged, contained or otherwise segregated from seed obtained from untreated plants are provided. In certain embodiments of such methods, the Methylobacterium strain is ISO10 (NRRL B-50938) or ISO20 (NRRL B-67743). In certain embodiments, the Methylobacterium strain has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium strain has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

Methods for identifying a Methylobacterium strain that increases the content of at least one mineral nutrient and/or at least one vitamin in a plant or plant part comprising: (i) treating a seed and/or a plant with at least a first Methylobacterium strain to obtain a treated seed and/or a treated plant; (ii) harvesting a plant part from a cultivated plant wherein the cultivated plant is grown from the treated seed or treated plant of step (i); (ii) harvesting a plant part from a cultivated control plant wherein the cultivated control plant was grown from an untreated control seed or untreated control plant; (iii) determining the content of at least one mineral nutrient and/or vitamin in the plant part from the cultivated plant and from the cultivated control plant; and, (iv) selecting a Methylobacterium strain that increases the content of at least one mineral nutrient or vitamin in the cultivated plant or a plant part of the cultivated plant in comparison to the content of the at least one mineral nutrient or vitamin in the cultivated control plant or plant part are provided.

Methods of identifying a Methylobacterium strain that increases the content of at least one mineral nutrient or vitamin in a plant or plant part comprising: (i) treating a first seed or plant with at least a first Methylobacterium strain and a second seed or plant part with a second Methylobacterium strain, (ii) harvesting a plant part from a plant grown from the first seed or plant, from a plant grown from the second seed or plant, and from a plant grown from a control seed or from a control plant; (iii) analyzing a plant part harvested from the plant grown from the first seed or plant, from the plant grown from the second seed or plant, and from the plant grown from the control seed or plant to determine the content of at least one mineral nutrient and/or vitamin, and (iv) selecting the Methylobacterium strain that provides the greatest increases in the content of the at least one mineral nutrient and/or vitamin in comparison to a plant part from the plant grown from the control seed, wherein the selected Methylobacterium strain is either the first Methylobacterium strain or the second Methylobacterium strain are provided.

Methods of producing a food or feed with increased mineral nutrient, vitamin, crude fat, oil and/or protein content comprising incorporating into the food or feed a processed or unprocessed food ingredient obtained from a cultivated plant or plants grown from Methylobacterium-treated seeds, plants or plant parts, thereby obtaining a food or feed with increased mineral nutrient, vitamin, oil and/or protein content are provided. In certain embodiments of such methods, the Methylobacterium strain is ISO10 (NRRL B-50938) or ISO20 (NRRL B-67743). In certain embodiments, the Methylobacterium strain has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium strain has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

A plant, or plant part having increased mineral nutrient, crude fat, seed oil, and/or protein content, wherein said plant or plant part is harvested from a cultivated plant grown from a Methylobacterium-treated seed, plant or plant part, wherein said Methylobacterium provides for increased mineral nutrient, crude fat, seed oil, and/or protein content. In certain embodiments, the Methylobacterium is ISO10 (NRRL B-50938) or ISO20 (NRRL B-67743). In certain embodiments, the Methylobacterium has chromosomal genomic DNA having at least 99%, 99.9, 99.8, 99.7, 99.6%, or 99.5% sequence identity to chromosomal genomic DNA of ISO10 (NRRL B-50938) or ISO20 (NRRL B-67743). In certain embodiments, the Methylobacterium has genomic DNA comprising one or more polynucleotide marker fragments of at least 50, 60, 100, 120, 180, 200, 240, or 300 nucleotides of SEQ ID NOS: 25-27 or SEQ ID NOS: 71-73.

### BRIEF DESCRIPTION OF THE DRAWINGS/FIGURES

FIG. 1 is a schematic diagram of canola or rapeseed processing methods (Image by Heuzé V., AFZ; from Heuzé V., Tran G., Sauvant D., Lessire M., Lebas F., 2018. Rapeseed meal. Feedipedia, a program by INRA, CIRAD, AFZ and FAO. Published on the world wide web site “feedipedia.org/node/52”).

FIG. 2 is a schematic diagram of soybean seed processing methods (Image by Tran, H., and Heuzé V., AFZ; from Heuzé V., Tran G., Kaushik S., 2017. Soybean meal. Feedipedia, a program by INRA, CIRAD, AFZ and FAO. Published on the world wide web site “feedipedia.org/node/674”).

FIG. 3 shows a comparison of % nitrogen content of seed harvested from soybean plants treated with Methylobacterium ISO10 and untreated control seed (t=−2.4; p=0.042).

FIG. 4 shows a comparison of % crude protein of seed harvested from soybean plants treated with Methylobacterium ISO10 and untreated control seed (t=−2.24; p=0.055).

FIG. 5 shows a comparison of % crude fat content of seed harvested from soybean plants treated with Methylobacterium ISO10 and untreated control seed (t=−7.11; p<0.0001).

FIG. 6 shows a comparison of % magnesium content of seed harvested from soybean plants treated with Methylobacterium ISO10 and untreated control seed (t=−4.47; p=0.0021).

FIG. 7 shows a comparison of % potassium content of seed harvested from soybean plants treated with Methylobacterium ISO10 and untreated control seed (t=3.37; p=0.0098).

FIG. 8 shows a comparison of % boron content of seed harvested from soybean plants treated with Methylobacterium ISO10 and untreated control seed (t=2.08; p=0.071).

FIG. 9 shows a comparison of iron content of stage V1 leaf samples from soybean plants grown from seeds treated with Methylobacterium ISO10 and stage V1 leaf samples from soybean plants grown from untreated seeds.

FIG. 10 shows a comparison of phosphorus content of stage V2 leaf samples from corn plants grown from seeds treated with Methylobacterium ISO15 and of stage V2 leaf samples from corn plants grown from untreated seeds.

FIG. 11 shows a comparison of potassium content of stage V2 leaf samples from corn plants grown from seeds treated with Methylobacterium ISO15 and of stage V2 leaf samples from corn plants grown from untreated seeds.

FIG. 12 shows a comparison of iron content of stage V2 leaf samples from corn plants grown from seeds treated with Methylobacterium ISO15 and of stage V2 leaf samples from corn plants grown from untreated seeds.

FIG. 13 shows a comparison of manganese content of stage V2 leaf samples from corn plants grown from seeds treated with Methylobacterium ISO15 and of stage V2 leaf samples from corn plants grown from untreated seeds.

### DETAILED DESCRIPTION

### Definitions

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features or encompassing the items to which they refer while not excluding any additional unspecified features or unspecified items.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses. In certain compositions, a biological can comprise a mono-culture or co-culture of Methylobacterium, or a combination of Methylobacterium strains or isolates that have been separately cultured.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); or Methylobacterium organophilum.

As used herein, the phrase “crude fat” refers to a diethyl ether extractable components of a given material (e.g., seed, seed meal, food or feed). Crude fat may include, but not limited to, true fats and oils, fatty acid esters, compound lipids, fat-soluble vitamins and provitamins (e.g., carotenoids), waxes, resins, and essential oils.

As used herein, the phrase “oil content” as applied to a seed or any processed product or fraction thereof refers to the fraction or percentage of the total mass or weight of the seed, processed product, or fraction thereof that is oil. Oil content can be expressed as a percentage of the total dry or weight of the seed, processed product, or fraction thereof.

As used herein, the term “oil” or phrase “seed oil” refers to the combination of triglycerides comprising saturated and/or unsaturated fatty acids, free saturated fatty acids, and free unsaturated fatty acids. Such fatty acids can include alpha-linolenic acid (C-18:3), linoleic acid (C-18:2), oleic acid (C-18:1), stearic acid (C-18:0), heptadecanoic (C-17:0), and/or palmitic acid (C-16:0).

As used herein, the terms “include,” “includes,” and “including” are to be construed as at least having the features to which they refer while not excluding any additional unspecified features.

As used herein, the phrase “protein content” as applied to a seed or any processed product or fraction thereof refers to the fraction or percentage of the total mass or weight of the seed, processed product, or fraction thereof that is protein. Protein content can be expressed as a percentage of the total dry or wet weight of the seed, processed product, or fraction thereof.

Protein content can also be expressed as mass units of protein per mass units seed, processed product, or fraction thereof (e.g., grams protein per kilogram dry or wet weight of the seed, processed product, or fraction thereof).

As used herein “mineral nutrients” are micronutrients or macronutrients required or useful for plants or plant parts including for example, but not limited to, nitrogen (N), potassium (K), calcium (Ca), magnesium (Mg), phosphorus (P), and sulfur (S), and the micronutrients chlorine (Cl), Iron (Fe), Boron (B), manganese (Mn), zinc (Z), copper (Cu), molybdenum (Mo) and nickel (Ni).

As used herein, “vitamins” are organic compounds required in small amounts for normal growth and metabolism. Vitamins are important for human and/or animal growth and some vitamins have been reported to be beneficial to plants. Vitamins include but are not limited to vitamin A (including but not limited to all-trans-retinol, all-trans-retinyl-esters, as well as all-trans-beta-carotene and other provitamin A carotenoids), vitamin B1(thiamine), vitamin B2 (riboflavin), vitamin B3 (niacin), vitamin B5(pantothenic acid), vitamin B6 (pyridoxine), vitamin B7 (biotin), vitamin B9(folic acid or folate), vitamin B12 (cobalamins), vitamin C (ascorbic acid), vitamin D (calciferols), vitamin E (tocopherols and tocotrienols), and vitamin K (quinones).

As used herein, the phrase “seed lot” refers to a collection of two or more seeds from one or more plants. In certain embodiments, a seed lot includes a collection of more than 10, 50, 100, 500, 1000 or more seeds from one or more plants.

As used herein, the term “strain” shall include all isolates of such strain.

As used herein, “variant” when used in the context of a Methylobacterium isolate, refers to any isolate that has chromosomal genomic DNA with at least 99%, 99.9, 99.8, 99.7, 99.6%, or 99.5% sequence identity to chromosomal genomic DNA of a reference Methylobacterium isolate, such as, for example, a deposited Methylobacterium isolate provided herein. A variant of an isolate can be obtained from various sources including soil, plants or plant material, and water, particularly water associated with plants and/or agriculture. Variants include derivatives obtained from deposited isolates. Methylobacterium isolates or strains can be sequenced (for example as taught by Sanger et al. (1977), Bentley et al. (2008) or Caporaso et al. (2012)) and genome-scale comparison of the sequences conducted (Konstantinos et al. (2005)) using sequence analysis tools (for example, BLAST, as taught by Altschul et al. (1990)).

As used herein, “derivative” when used in the context of a Methylobacterium isolate, refers to any Methylobacterium that is obtained from a deposited Methylobacterium isolate provided herein. Derivatives of a Methylobacterium isolate include, but are not limited to, derivatives obtained by selection, derivatives selected by mutagenesis and selection, and genetically transformed Methylobacterium obtained from a Methylobacterium isolate. A “derivative” can be identified, for example based on genetic identity to the strain or isolate from which it was obtained and will generally exhibit chromosomal genomic DNA with at least 99%, 99.9, 99.8, 99.7, 99.6%, or 99.5% sequence identity to chromosomal genomic DNA of the strain or isolate from which it was derived.

As used herein, the term “cultivate” means to grow a plant. A cultivated plant can be one grown and raised on a large agricultural scale or on a smaller scale, including for example a single plant.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Various methods where Methylobacterium strains are used to increase the mineral nutrient, vitamin, oil and/or protein content in plants or plant parts harvested from plants grown from seed and/or plants comprising plant parts treated with the Methylobacterium are provided herein. In certain embodiments, Methylobacterium treatment of soil, a seed, a leaf, a fruit, a stem, a root, or a shoot can increase plant mineral nutrient, vitamin, crude fat, seed oil, and/or protein content in seeds or seed lots harvested from plants grown from the treated seed or plant comprising the treated plant part. In other embodiments, Methylobacterium treatment of soil, a seed, a leaf, a fruit, a stem, a root, or a shoot can increase the content of one or more mineral nutrients or vitamins in harvested plants or plant parts from plants grown from the Methylobacterium-treated plant parts or Methylobacterium-treated seeds provided herein. Methylobacterium soil treatments or applications can include, but are not limited to, in-furrow applications (e.g., before, during, and/or after seed deposition), soil drenches, distribution of granular or other dried formulations to the soil (e.g., before, during, and/or after seed deposition or plant growth). In certain embodiments, Methylobacterium treatment of a seed and/or plant can thus comprise any Methylobacterium soil treatment or application where the seed and/or plant is contacted and/or colonized by the Methylobacterium. Treatments or applications can include, but are not limited to, spraying, coating, partially coating, immersing, and/or imbibing the plant or plant parts with the Methylobacterium strains and compositions comprising the same provided herein. In certain embodiments, soil, a seed, a leaf, a fruit, a stem, a root, a tuber, or a shoot can be sprayed, immersed and/or imbibed with a liquid, semi-liquid, emulsion, or slurry of a composition provided herein. Such seed immersion or imbibition can be sufficient to provide for increased crude fat, seed oil, and/or protein content in a treated plant or plant grown from a treated seed in comparison to an untreated plant or plant grown from an untreated seed. In certain embodiments, plant seeds can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30 degrees Centigrade or at about 20 to about 25 degrees Centigrade. In certain embodiments, seed imbibition and/or immersion can be performed with gentle agitation. Seed treatments can be effected with both continuous and/or a batch seed treaters. In certain embodiments, the coated seeds can be prepared by slurrying seeds with a coating composition comprising a Methylobacterium strain that increases mineral nutrient, vitamin, crude fat, seed oil, and/or protein content and air-drying the resulting product. Air-drying can be accomplished at any temperature that is not deleterious to the seed or the Methylobacterium, but will typically not be greater than 30 degrees Centigrade. The proportion of coating that comprises the Methylobacterium strain includes, but is not limited to, a range of 0.1 to 25% by weight of the seed or other plant part, 0.5 to 5% by weight of the seed or other plant part, and 0.5 to 2.5% by weight of the seed or other plant part. In certain embodiments, a solid substance used in the seed coating or treatment will have a Methylobacterium strain that increases mineral nutrient, vitamin, crude fat, seed oil, and/or protein content adhered to a solid substance by being grown in biphasic media comprising the Methylobacterium strain, solid substance, and liquid media. Methods for growing Methylobacterium in biphasic media include those described in U.S. Pat. No. 9,181,541, which is specifically incorporated herein in its entirety. In certain embodiments, compositions suitable for treatment of a seed or plant part with a Methylobacterium strain that increases mineral nutrient, vitamin, crude fat, seed oil, and/or protein content can be obtained by the methods provided in US Patent Application No. US 20160120188, which is specifically incorporated herein in its entirety. Various seed treatment compositions and methods for seed treatment disclosed in U.S. Pat. Nos. 5,106,648, 5,512,069, and 8,181,388 are incorporated herein by reference in their entireties and can be adapted for treating seeds with compositions comprising a Methylobacterium strain.

In certain embodiments, the composition used to treat the seed or plant part can contain a Methylobacterium strain and an agriculturally acceptable excipient. Agriculturally acceptable excipients include, but are not limited to, woodflours, clays, activated carbon, diatomaceous earth, fine-grain inorganic solids, calcium carbonate and the like. Clays and inorganic solids that can be used with the include, but are not limited to, calcium bentonite, kaolin, china clay, talc, perlite, mica, vermiculite, silicas, quartz powder, montmorillonite and mixtures thereof.

Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in U.S. Pat. No. 8,181,388 can be adapted for use with compositions comprising a suitable Methylobacterium strain. In certain embodiments, the seed and/or seedling is exposed to the composition by providing the Methylobacterium strain in soil in which the plant or a plant arising from the seed are grown, or other plant growth media in which the plant or a plant arising from the seed are grown. Examples of methods where the Methylobacterium strain is provided in the soil include in furrow applications, soil drenches, and the like.

In certain embodiments, a Methylobacterium strain that increases mineral nutrient, vitamin, crude fat, seed oil, and/or protein content used to treat a given species of seed or plant part can be a Methylobacterium strain that was isolated from a different plant species and is thus heterologous to the treated plant or plant part. Non-limiting examples of treatments of plant seed or other plant parts with a heterologous Methylobacterium include treatments of soybean, Brassica sp., sunflower, cotton, flax, or peanut seeds and parts with a Methylobacterium strain that was isolated from a plant other than a soybean, Brassica sp., sunflower, cotton, flax, or peanut plant, respectively.

Non-limiting examples of treatments of plant seed or other plant parts with a Methylobacterium providing for increased content of one or more mineral nutrients, vitamins, crude fat, seed oil, and/or protein in a harvested plant part include treatments of corn, soybean, Brassica sp. (e.g., B. napus, B. rapa, B. juncea), alfalfa, rice, rye, wheat, barley, oats, sorghum, millet (e.g., pearl millet (Pennisetum glaucum)), proso millet (Panicum miliaceum), foxtail millet (Setaria italica), finger millet (Eleusine coracana), sunflower, safflower, tobacco, potato, peanuts, cotton, species in the genus Cannabis including, but not limited to, Cannabis sativa and industrial hemp varieties, sweet potato (Ipomoea batatus), cassava, coffee, coconut. pineapple, citrus trees, cocoa, tea, date palm, banana, apple, pear, grape, berry plants (including, but not limited to blackberry, raspberry, strawberry or blueberry plants), avocado, fig, guava, kiwi, mango, olive, papaya, cashew, macadamia, almond, sugar beets, sugarcane, tomatoes, peppers, carrots, celery, lettuce, green beans, lima beans, peas, lentils, cucurbits (including, but not limited to cucumber, cantaloupe, melons, squash, pumpkin, and zucchini). In other embodiments, treated plants include ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar) and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass). In certain embodiments, a Methylobacterium strain that increases mineral nutrient, vitamin, crude fat, seed oil, and/or protein content used to treat a given cultivar or variety of seed, plant or plant part can be a Methylobacterium strain that was isolated from a different plant species, or a different cultivar or variety of the plant species being treated, and is thus non-resident to the treated plant or plant part.

Plant parts that have increased levels of one or more mineral nutrients, vitamins, crude fat, seed oil, and/or protein as the result of treatment with Methylobacterium as provided herein include, but are not limited to, leaves, stems, flowers, roots, seeds, fruit, tubers, coleoptiles, and the like.

In some embodiments, a plant part having increased levels of one or more mineral nutrients, vitamins, crude fat, seed oil, and/or protein is a plant seed. Of particular interest for treatments of plant seed or other plant parts with Methylobacterium providing for increases in mineral nutrients, vitamins, crude fat, seed oil, and/or protein in harvested plant seeds are treatments of soybean, Brassica sp., sunflower, cotton, flax, or peanut seeds and parts. In some embodiments, such treatments are with a Methylobacterium strain that was isolated from a different species, cultivar or variety compared to the plant being treated.

In certain embodiments, a manufactured combination composition comprising two or more Methylobacterium strains can be used to treat a seed or plant part in any of the methods provided herein. Such manufactured combination compositions can be made by methods that include harvesting monocultures of each Methylobacterium strain and mixing the harvested monocultures to obtain the manufactured combination composition of Methylobacterium. In certain embodiments, the manufactured combination composition of Methylobacterium can comprise Methylobacterium isolated from different plant species or from different cultivars or varieties of a given plant.

In certain embodiments, an effective amount of the Methylobacterium strain or strains that increase mineral nutrient, vitamin, crude fat, seed oil, and/or protein content used in treatment of seeds or plant parts is a composition having a Methylobacterium titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an effective amount of the Methylobacterium strain or strains that increase mineral nutrient, vitamin, crude fat, seed oil, and/or protein content used in treatment of seeds or plant parts is a composition with the Methylobacterium at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an effective amount of the Methylobacterium strain or strains that increase mineral nutrient, vitamin, crude fat, seed oil, and/or protein content used in treatment of seeds or plant parts is a composition with the Methylobacterium at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of the composition. In certain embodiments, an effective amount of a composition provided herein that is sufficient to provide for increased mineral nutrient, vitamin, crude fat, seed oil, and/or protein content can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles that comprise a solid substance wherein a mono-culture or co-culture of crude fat, seed oil, and/or protein content enhancing Methylobacterium strain or strains is adhered thereto. In certain embodiments, an effective amount of a composition provided herein that is sufficient to provide for increased mineral nutrient, vitamin, crude fat, seed oil, and/or protein content to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains that increase mineral nutrient, vitamin, crude fat, seed oil, and/or protein content adhered to a solid substance is provided therein or grown therein. In certain embodiments, an effective amount of a composition provided herein that is sufficient to provide for increased mineral nutrient, vitamin, crude fat, seed oil, and/or protein content to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL of in a composition comprising an emulsion wherein a mono-culture or co-culture of a Methylobacterium strain or strains that increase mineral nutrient, vitamin, crude fat, seed oil, and/or protein content is provided therein or grown therein. In certain embodiments, any of the aforementioned compositions comprising a mono-culture or co-culture of a Methylobacterium strain or strains that increase mineral nutrient, vitamin, crude fat, seed oil, and/or protein content can further comprise a mono- or co-culture of Rhizobium and/or Bradyrhizobium.

An effective amount of a Methylobacterium strain or strains that increase mineral nutrient, vitamin, crude fat, seed oil, and/or protein content provided in a treatment of a seed or plant part is an amount that results in an increase in the mineral nutrient, vitamin, crude fat, seed oil, and/or protein content of seed or other plant parts harvested from a plant grown from the treated seed or plant comprising the treated plant part. In certain embodiments, an effective amount of a Methylobacterium strain or strains that increase mineral nutrient, vitamin, crude fat, seed oil, and/or protein content provided in a treatment of a seed or plant part is at least about 10¹, 10⁴, 10⁵, or 10⁶ CFU per seed or treated plant part. In certain embodiments, an effective amount of Methylobacterium provided in a treatment of a seed or plant part is at least about 10³, 10⁴, 10¹, or 10⁶ CFU to about 10⁷, 10⁸, 10⁹, or 10¹¹ CFU per seed or treated plant part. In certain embodiments, the effective amount of Methylobacterium provided in a treatment of a seed or plant part is an amount where the CFU per seed or treated plant part will exceed the number of CFU of any resident naturally occurring Methylobacterium strain by at least 5-, 10-, 100-, or 1000-fold. In certain embodiments, the effective amount of Methylobacterium provided in a treatment of a seed or plant part is an amount where the CFU per seed or treated plant part will exceed the number of CFU of any resident naturally occurring Methylobacterium by at least 2-, 3-, 5-, 8-, 10-, 20-, 50-, 100-, or 1000-fold.

Methylobacterium sp. strains are useful in certain methods provided herein. In one embodiment, the Methylobacterium strain includes certain Methylobacterium strains obtained from Methylobacterium species that include M. gregans, M. komagatae, and M. radiotolerans. Non-limiting examples of other Methylobacterium sp. strains of use in certain methods provided herein are disclosed in Table 1. Other Methylobacterium sp. strains useful in certain methods provided herein include variants of the Methylobacterium sp. strains disclosed in Table 1.

TABLE 1
Methylobacterium sp. strain

| | Isolate | | USDA ARS | |
|---|---|---|---|---|
| Deposit Identifier | No. | NLS NO. | NRRL No.¹ | Strain Source: Obtained from: |
| Methylobacterium sp. #1 | ISO01 | NLS0046 | NRRL B-50929 | a soybean plant grown in Saint |
| | | | | Louis County, Missouri, USA |
| Methylobacterium sp. #2 | ISO02 | NLS0020 | NRRL B-50930 | a weed grown in Saint Louis |
| | | | | County, Missouri, USA |
| Methylobacterium sp. #3 | ISO03 | NLS0017 | NRRL B-50931 | a mint plant grown in Saint Louis |
| | | | | County, Missouri, USA |
| Methylobacterium sp. #4 | ISO04 | NLS0042 | NRRL B-50932 | a soybean plant grown in Saint |
| | | | | Louis County, Missouri, USA |
| Methylobacterium sp. #5 | ISO05 | NLS0089 | NRRL B-50933 | a broccoli plant grown in Saint |
| | | | | Louis County, Missouri, USA |
| Methylobacterium sp. #6 | ISO06 | NLS0068 | NRRL B-50934 | a corn plant grown in Saint Louis |
| | | | | County, Missouri, USA |
| Methylobacterium sp. #7 | ISO07 | NLS0065 | NRRL B-50935 | a corn plant grown in Saint Louis |
| | | | | County, Missouri, USA |
| Methylobacterium sp. #8 | ISO08 | NLS0069 | NRRL B-50936 | a corn plant grown in Saint Louis |
| | | | | County, Missouri, USA |
| Methylobacterium sp. #9 | ISO09 | NLS0062 | NRRL B-50937 | a corn plant grown in Saint Louis |
| | | | | County, Missouri, USA |
| Methylobacterium sp. #10 | ISO10 | NLS0064 | NRRL B-50938 | a corn plant grown in Saint Louis |
| | | | | County, Missouri, USA |
| Methylobacterium sp. #11 | ISO11 | NLS0021 | NRRL B-50939 | a lettuce plant grown in Saint |
| | | | | Louis County, Missouri, USA |
| Methylobacterium sp. #12 | ISO12 | NLS0066 | NRRL B-5 0940 | a corn plant grown in Saint Louis |
| | | | | County, Missouri, USA |
| Methylobacterium sp. #13 | ISO13 | NLS0037 | NRRL B-50941 | a tomato plant grown in Saint |
| | | | | Louis County, Missouri, USA |
| Methylobacterium sp. #14 | ISO14 | NLS0038 | NRRL B-50942 | a tomato plant grown in Saint |
| | | | | Louis County, Missouri, USA |
| Methylobacterium #15 | ISO15 | NLS0044 | NRRL B-67339 | a soybean plant grown in Saint |
| | | | | Louis County, Missouri, USA |
| Methylobacterium #16 | ISO16 | NLS0109 | NRRL B-67340 | a yucca plant grown in Saint |
| | | | | Louis County, Missouri, USA |
| Methylobacterium sp | ISO18 | NLS0648 | NRRL B-67741 | a Dionaea muscipula plant |
| (#18) | | | | (Venus fly trap) grown in St. |
| | | | | Charles, MO. |
| Methylobacterium sp | ISO19 | NLS0662 | NRRL B-67742 | an Orchidaceae spp. plant |
| (#19) | | | | (orchid) grown in Saint Louis |
| | | | | County, Missouri, USA |
| Methylobacterium sp | ISO20 | NLS0807 | NRRL B-67743 | a tomato plant grown in Saint |
| (#20) | | | | Louis County, Missouri, USA |
| Methylobacterium sp | ISO26 | NLS0610 | Deposited under | A Lagerstroemia indica (crape |
| (#26) | | | Budapest Treaty | myrtle) plant grown in Saint |
| | | | with the NRRL, | Louis County, Missouri, USA |
| | | | Peoria, IL, USA | |
| | | | on Nov. | |
| | | | 26, 2019; | |
| | | | NRRLB-67895 | |

¹Deposit number for strain deposited with AGRIGULTURAL RESEARCH SERVICE CULTURE COLLECTION (NRRL) of the National Center Agricultural Utilization Research, Agricultural Research Service, U.S. Department of Agriculture, 1815 North University Street, Peoria, Illinois 61604 U.S.A. under the terms of the Budapest Treaty on the International Recoginition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Variants of a Methylobacterium isolate listed in Table 1 include isolates obtained therefrom by genetic transformation, mutagenesis and/or insertion of a heterologous sequence. In some embodiments, such variants are identified by the presence of chromosomal genomic DNA with at least 99%, 99.9, 99.8, 99.7, 99.6%, or 99.5% sequence identity to chromosomal genomic DNA of the strain from which it was derived. In certain embodiments, such variants are distinguished by the presence of one or more unique DNA sequences that include: (i) a unique sequence of SEQ ID NOs: 1 to 3, SEQ ID NOs: 13 to 15, SEQ ID NOs: 25 to 27, SEQ ID NOs: 37 to 39, SEQ ID NOs: 49 to 51, and SEQ ID NOs: 61 to 73; or (ii) sequences with at least 98% or 99% sequence identity across the full length of SEQ ID NOs: 1 to 3, SEQ ID NOs: 13 to 15, SEQ ID NOs: 25 to 27, SEQ ID NOs: 37 to 39, SEQ ID NOs: 49 to 51, SEQ ID NOs: 61 to 73, and SEQ ID Nos:74 to 76.

Co-assigned patent applications disclose additional specific uses of certain Methylobacterium strains of Table 1 such as: increasing corn yield (US20160295868); improving lettuce cultivation (U.S. Pat. No. 10,212,939); improving tomato growth (US20170086464); improving soy yield (US2016/0302423); improving fruit production (U.S. Pat. No. 10,111,438); controlling corn rootworm (US 20170238553); controlling root lesion nematodes (US20170164618); controlling root knot nematodes (U.S. Pat. No. 10,098,353); and controlling fungal disease (US20180295841 and WO2018106899) are each incorporated herein by reference in their entireties. In certain embodiments of the methods provided herein, the Methylobacterium strain or strains used to treat a seed and/or a plant part are selected from the group consisting of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B-50940), ISO13 (NRRL B-50941), ISO14 (NRRL B-50942), ISO16 (NRRL B-67340), ISO18(NRRL B-67741), ISO19 (NRRL B-67742), ISO20 (NRRL B-67743), variants thereof, or any combination thereof. In certain embodiments, one or more of the Methylobacterium strains used in the methods can comprise total genomic DNA (chromosomal and plasmid DNA) or average nucleotide identity (ANI) with at least 99%, 99.9, 99.8, 99.7, 99.6%, or 99.5% sequence identity or ANI to total genomic DNA of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B-50940), ISO13 (NRRL B-50941), ISO14 (NRRL B-50942), ISO16 (NRRL B-67340), ISO18(NRRL B-67741), ISO19 (NRRL B-67742), or ISO20 (NRRL B-67743). In certain embodiments, the percent ANI can be determined as disclosed by Konstantinidis et al., 2006. In certain embodiments of the methods provided herein, the Methylobacterium strain or strains used to treat a seed and/or a plant part is not the strain identified as either ISO10 or NLS0064 which was deposited under the NRRL accession No. NRRL B-50938. In certain embodiments of the methods provided herein, the strain identified as either ISO10 or NLS0064 which was deposited under the NRRL accession No. NRRL B-50938 is not used. In certain methods provided herein for identifying Methylobacterium that can improve crude fat, seed oil, and/or protein content, any of the aforementioned strains set forth in Table 1 can be used as a control or reference standard for comparison to one or more new test or candidate Methylobacterium isolates. In certain embodiments, the strain identified as either ISO10 or NLS0064 which was deposited under the NRRL accession No. NRRL B-50938 is used as a control or reference standard for comparison to one or more new test or candidate Methylobacterium isolates in a method of identifying a new Methylobacterium that can improve crude fat, seed oil, and/or protein content.

In certain embodiments of the methods provided herein, seeds and/or plant parts are treated with both a Methylobacterium strain and at least one additional component. In some embodiments an additional component can be an additional active ingredient, for example, a pesticide or a second biological. In certain embodiments, the pesticide can be an insecticide, a fungicide, an herbicide, a nematicide or other biocide. The second biological could be a strain that improves yield or controls an insect, pest, fungi, weed, or nematode.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides, include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins, Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a Methylobacterium strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, the composition or method disclosed herein may comprise an additional active ingredient, which may be a second biological. The second biological could be a biological control agent, other beneficial microorganisms, microbial extracts, natural products, plant growth activators or plant defense agent. Non-limiting examples of the second biological could include bacteria, fungi, beneficial nematodes, and viruses. In certain embodiments, the second biological can be a Methylobacterium. In certain embodiments, the second biological is a Methylobacterium listed in Table 1. In certain embodiments, the second biological can be a Methylobacterium selected from M. gregans, M. radiotolerans, M. extorquens, M. populi, M. salsuginis, M. brachiatum, and M. komagatae.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments, the second biological is a Rhizobium or Bradyrhizobium, which is beneficial to growth of plants grown from treated seed and/or a plant comprising a treated plant part. Treatments of leguminous plant seeds or other plant parts including soybean and peanut with both a Methylobacterium strain or strains and the Rhizobium or Bradyrhizobium can result in an improvement in crude fat, seed oil, and/or protein content of seed harvested from a plant grown from the treated seed and/or plant parts in comparison to a control plant grown from seed and/or plant parts treated with just the Rhizobium or Bradyrhizobium (i.e., not treated with the Methylobacterium) and/or to a control plant grown from seed and/or plant parts that were not treated with a Methylobacterium strain and that were not treated with the Rhizobium or Bradyrhizobium.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium. In particular embodiments, the fungus is Beauveria bassiana, Coniothyrium minitans, Gliocladium vixens, Muscodor albus, Paecilomyces lilacinus, or Trichoderma polysporum.

In further embodiments the second biological can be plant growth activators or plant defense agents including, but not limited to harpin, Reynoutria sachalinensis, jasmonate, lipochitooligosaccharides, and isoflavones.

In further embodiments, the second biological can include, but are not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the second biological can be provided in the composition in the form of a spore.

Seed lots comprising at least about 50%, 70%, 80%, 90%, 95%, 98%, or 99% of seeds harvested from mature plants grown from seeds and/or plants treated with an effective amount of a Methylobacterium strain or strains are provided herein. Such seed lots can exhibit increased crude fat, seed oil, and/or protein content and are thus useful in a variety of processes. In certain embodiments, such seed lots can be used as feedstock in methods of extracting oil and/or protein from seed. It is thus anticipated that the yield of oil and/or protein will be increased by use of the seed lots provided herein as feedstock in comparison to methods where seed lots obtained from commodity seed are used as feedstock. Such commodity seed would in general comprise seed harvested from mature plants grown from seeds and/or plants that were not treated with an effective amount of a Methylobacterium strain or strains. In certain embodiments, such seed lots can be used in methods of manufacturing a food or feed ingredient to obtain a food or feed ingredient with increased oil and/or protein content. In certain embodiments, the seed lot will comprise at least about 10, 20, 50, 100, 500, or 1000 seed. In certain embodiments, the seed lot will comprise at least about 10, 20, 50, 100, 500, or 1000 pounds or kilograms of seed. In certain embodiments, the seed lot will comprise at least about 100, 200, 500, 1000, 5000, or 10,000 bushels of seed. Seed lots can be packaged or contained in a bag, box, or other packaging suitable for storing and/or shipping. In certain embodiments, the seed lot can be packaged, stored, and/or shipped in a silo bag. In certain embodiments, such silo bags can comprise a triple layer of thick laminated extruded plastic (polyethylene) about 230-235 microns thick, where the two outer layers are white with a UV protective coating while the inner layer in contact with the seed is black (Ileleji, K. Use of Silo Bags for Commodity Grain Storage in Indiana, 2014; on the https internet site “extension.purdue.edu/article/6963”). In certain embodiments, a silo bag filled with a seed lot will comprise a tube of about 8 to 12 ft in diameter and up to about 200 ft in length and can hold about 8,000 to 12,000 bushels of seed. In certain embodiments, any of the aforementioned packaged seed lots can comprise a tag or label useful of identifying and tracking the seed lot. Such tags or labels can comprise a barcode, an RFID tag, or other identifiers. In certain embodiments, the tag or label will identify the seed lot as comprising at least about 50%, 70%, 80%, 90%, 95%, 98%, or 99% of seeds harvested from mature plants grown from seeds and/or plants treated with an effective amount of a Methylobacterium strain or strains. In certain embodiments, the tag or label will identify the seed lot as comprising at least about 50%, 70%, 80%, 90%, 95%, 98%, or 99% of seeds having an increased crude fat, seed oil, and/or protein content in comparison to commodity seed.

In certain embodiments of the methods provided herein, the seed lots harvested from mature plants grown from seeds and/or plants treated with an effective amount of a Methylobacterium strain or strains are segregated from other seed lots comprising commodity seed that has not been uniformly subjected to such Methylobacterium treatments. Methods for achieving such segregation include: (1) planting and/or treating defined areas of a field with Methylobacterium-strain treated seed and/or with a Methylobacterium treatment (e.g., in furrow and/or foliar applications, soil drenches, and or any combination thereof); (2) separating the defined areas of a field from adjacent untreated areas with a unplanted or other border which permits separate harvest of seed from the mature plants; and/or (3) use of global position system (GPS) coordinates in the planting, treating, and or harvesting machinery to facilitate harvest of seed from mature plants grown from seeds and/or plants treated with an effective amount of a Methylobacterium strain or strains. Segregation and transfer of the seed lot to an oil and/or protein extraction plant, a food or feed ingredient manufacturing facility, or a food or feed manufacturing facility are also facilitated by use packaging, labelling, or tagging systems such as those described above.

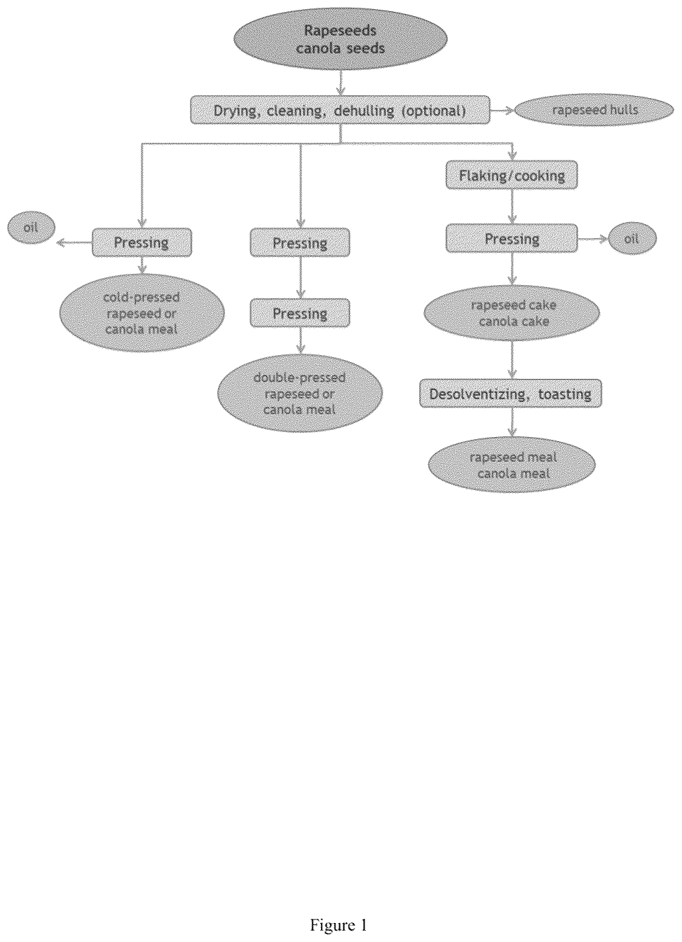

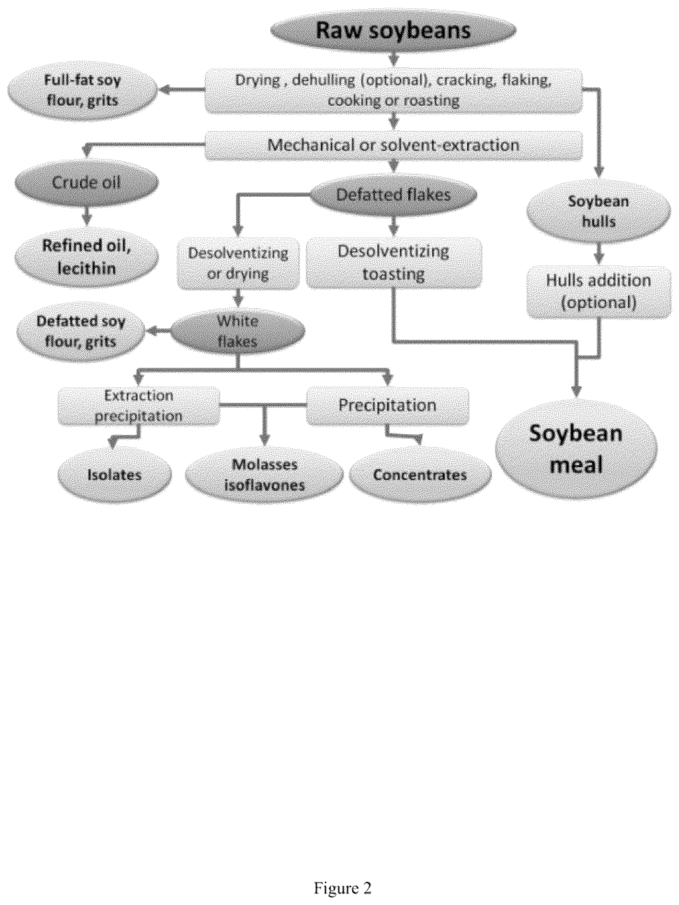

Seed lots comprising at least 50%, 70%, 80%, 90%, or 95% of the seeds harvested from mature plants grown from seeds and/or plants treated with an effective amount of a Methylobacterium strain or strains can also be used to obtain seed oil and/or seed protein-enriched fractions, to increase yield of protein- or oil-containing fractions from seed lots, to manufacture food or feed ingredients, or to manufacture food or feed. Schematic diagrams of certain seed processing methods are depicted in FIG. 1 (for Brassica sp. seeds including rapeseed and canola seed) and FIG. 2 (for soybean). FIGS. 1 and 2 are solely illustrative, as any seed processing methods can be employed. Such processes depicted in FIGS. 1 and 2 are also illustrative of processes which can be adapted for use with other oil seeds such as sunflower, flax, cotton, peanut, linseed, and the like. For example, cotton seed would be delinted in most instances where cottonseed was used as a feedstock.

Food or feed ingredients obtained from the seed lots provided herein include crude or refined seed oil, pressed seed cake, pressed or double pressed seed meal, defatted or partially defatted seed meal or flakes, solvent extracted seed meal, desolventized seed meal, expandate resulting from passing processed seed materials through an expander, extrudate resulting from passage of processed seed materials through an extruder, and the like. In certain embodiments, the feed ingredients can comprise pressed seed cake or meal, defatted or partially defatted seed meal, solvent extracted seed meal, desolventized seed meal, expandate, or extrudate, which has been pelletized. An oil-enriched fraction obtained by the methods provided herein can thus comprise a crude oil (e.g., oil obtained from seed by a mechanical pressing or expelling of seed or a processed product thereof), a seed oil containing solvent fraction, or a desolventized oil obtained from a seed oil containing solvent fraction. Seed oil containing solvent fractions can be obtained by extraction of processed seed lot products with solvents such as hexane (e.g., mixed hexanes). Seed oil can also be prepared by supercritical fluid extraction (SFE; e.g., supercritical CO₂) of processed seed lot products. Supercritical fluid extraction (e.g., supercritical CO₂) of processed seed lot products methods described in the literature (e.g., Jokid et al., 2012) can be adapted for use with feedstock comprising seed lots provided herein. Processed seed lot products that can be subject to mechanical pressing or expelling and/or to solvent extraction include dehulled, delinted, crushed, macerated, and/or ground seed, expandate, and extrudate. Expandate is obtained by passage of seed materials through expanders, which subject processed seed material to high pressure, shear forces, and high temperatures for short time intervals of about a minute through steam injection. Commercial expanders include an Anderson oilseed expander (Anderson International, Stow, OH, USA), Andritz Expander (Andritz, Graz, Austria), Kahl expander (Amandus Kahl USA, Atlanta, GA), and the like. As used herein, an extrudate is defined as a material that is obtained by subjecting a seed material to pressure and/or shear forces without steam injection. A protein-enriched fraction produced by the methods provided herein can thus include pressed seed cake, pressed or double pressed seed meal, defatted or partially defatted seed meal or flakes, solvent extracted seed meal, desolventized seed meal, or expandate. A protein-enriched fraction can also include an aqueous fraction obtained by extracting dehulled, delinted, crushed, macerated, and/or ground seed, expandate, pressed seed cake, pressed or double pressed seed meal, defatted or partially defatted seed meal or flakes, solvent extracted seed meal, desolventized seed meal, and/or extrudate with water, a buffered aqueous solution, an acidic aqueous solution, a basic aqueous solution, or a salt-containing solution.

Processed or unprocessed food or feed ingredients obtained from seed lots comprising at least 50%, 70%, 80%, 90%, or 95% of the seeds harvested from mature plants grown from seeds and/or plants treated with an effective amount of a Methylobacterium strain or strains can be used as ingredients in methods of making a food or feed with improved mineral nutrient, vitamin, oil and/or protein content. In certain embodiments, such processed food or feed ingredients can comprise dehulled, delinted, crushed flaked, defatted or partially defatted seed meal or seed cake, non-defatted seed meal from the seed lot or an oil and/or a protein enriched fraction from the seed lot. In certain embodiments, such unprocessed food ingredients can comprise whole seed from the seed lot. In certain embodiments, whole seed can be combined with silage, seed meal, additional vitamins (e.g., vitamins A, D, and/or E), additional mineral nutrients (e.g., calcium, sodium, iron, magnesium, manganese, cobalt, selenium, dyes, and/or amino acids (e.g., methionine and/or lysine) to provide animal feed. Such food or feed ingredients obtained from the seed lot can be incorporated into a food or feed by steps comprising at least one of combining, mixing, heating, extruding, pelletizing, molding, and or forming that food or feed ingredient into a food or feed product. In certain embodiments, the food or feed ingredients obtained from the seed lot can be incorporated into a food or feed with another food or feed ingredient by steps comprising at least one of combining, mixing, heating, extruding, pelletizing, molding, and or forming the food or feed ingredients into a food or feed product. Additional food or feed ingredients that can be incorporated into the food of feed product in combination with food or feed ingredients obtained from the seed lot include starches, hydrolyzed starches, modified starches, sugars, fiber, emulsifiers, flavorants, additional vitamins (e.g., vitamins A, D, and/or E), additional mineral nutrients (e.g., calcium, sodium, iron, magnesium, manganese, cobalt, selenium, dyes, amino acids (e.g., methionine and/or lysine), antimicrobial agents, and the like. Methods and compositions for making food-, pet food- or feed compositions comprising pellets, emulsions, fat compositions, and the like disclosed in U.S. Patent Appl. Pub. Nos. US20180125101, US20180103654, and/or US20170112159, which are each incorporated herein in their entireties, can be adapted for use with the food and feed ingredients provided herein to make food or feed products.

Seed lots comprising at least 50%, 70%, 80%, 90%, or 95% of the seeds harvested from mature plants grown from seeds and/or plants treated with an effective amount of a Methylobacterium strain or strains can be used as feedstock to increase yield of oil in oil-enriched fractions in comparison to the yield obtained when control seed lots are used as feedstock. In this embodiment, oil yield is defined as the amount of oil in an oil-enriched fraction obtained per unit of seed lot feedstock used in an oil extraction process. In certain embodiments, oil yield can be expressed as the mass, or weight of oil in an oil-enriched fraction obtained from an oil extraction process per kilogram seed used in the process. In certain embodiments, oil yield can be expressed as the mass, or weight of oil in an oil-enriched fraction obtained from an oil extraction process per seed or per bushel of seed used in the process. In other embodiments, oil yield can be expressed as a volume of oil obtained or present in an oil-enriched fraction per kilogram seed used in the process. Separation steps that can be used in an oil extraction process to obtain an oil-enriched fraction can include mechanical pressing or expelling of seed or a processed seed product (e.g., non-defatted seed meal, flakes, expandate, or extrudate) to obtain a crude oil fraction. Separation steps that can be used to obtain an oil-enriched fraction can also include solvent or SFE extraction of a processed seed product (e.g., non-defatted seed meal, flakes, expandate, or extrudate) to obtain an oil-enriched fraction. In certain embodiments, seed oil yield can be improved relative to controls even with reduced energy inputs into an oil extraction process by using the aforementioned seed lots as feedstock. In certain embodiments, seed oil yield can be improved when temperatures of oil extraction processes (e.g., pressing, expansion, or extrudation) are reduced by at least 1%, 2%, 5%, 10%, or 20% in comparison to processes where a control seed lot (e.g., a seed lot obtained from plants or plants grown from seeds that were not treated with Methylobacterium) is processed under reduced temperatures. In certain embodiments, seed oil yield can be improved when temperatures of oil extraction processes (e.g., pressing, expansion, or extrudation) are reduced by about 1%, 2%, or 5%, to 10%, 20%, 30%, 40%, or 50% in comparison to processes where a control seed lot (e.g., a seed lot obtained from plants or plants grown from seeds that were not treated with Methylobacterium) is processed under the same reduced temperatures. In certain embodiments, seed oil yield can be improved when energy inputs into an oil extraction process are reduced by at least 1%, 2%, 5%, 10%, or 20% in comparison to processes where a control seed lot (e.g., a seed lot obtained from plants or plants grown from seeds that were not treated with Methylobacterium) is processed under the same reduced energy input conditions. In certain embodiments, seed oil yield can be improved when energy inputs into an oil extraction process are reduced by about 1%, 2%, or 5%, to about 10%, 20%, 30%, 40%, or 50% in comparison to processes where a control seed lot (e.g., a seed lot obtained from plants or plants grown from seeds that were not treated with Methylobacterium) is processed under the same reduced energy input conditions.

Seed lots comprising at least 50%, 70%, 80%, 90%, or 95% of the seeds harvested from mature plants grown from seeds and/or plants treated with an effective amount of a Methylobacterium strain or strains can be used as feedstock to increase yield of protein in protein-enriched fractions in comparison to the yield obtained when control seed lots are used as feedstock in a protein extraction process. In this embodiment, protein yield is defined as the amount of protein in a protein-enriched fraction obtained per unit of seed lot feedstock used in a protein extraction process. In certain embodiments, protein yield can be expressed as the mass or weight of protein in a protein-enriched fraction obtained in a protein extraction process per kilogram seed used in the process. Separation steps that can be used to obtain a protein-enriched fraction can include delinting and/or dehulling of seed to obtain a protein-enriched fraction comprising delinted and/or dehulled whole seed. Separation steps that can be used to obtain a protein-enriched fraction can also include grinding, macerating, flaking, and/or drying of whole, delinted, or dehulled seed to obtain a protein enriched product comprising seed meal, flakes, and the like which can be non-defatted, defatted, or partially defatted. Separation steps that can be used to obtain a protein-enriched fraction can include mechanical pressing or expelling of seed or a processed seed product (e.g., non-defatted seed meal, flakes, expandate, or extrudate) to obtain a protein-enriched fraction comprising pressed seed meal or a seed cake. Separation steps that can be used to obtain a protein-enriched fraction can also include solvent or SFE extraction of a processed seed product (e.g., non-defatted seed meal, flakes, expandate, or extrudate) to obtain a protein-enriched fraction comprising defatted or partially defatted seed meal, flakes, expandate, or extrudate. In certain embodiments, seed protein yield can be improved relative to controls even with reduced energy inputs into a protein-enrichment process by using the aforementioned seed lots as feedstock. In certain embodiments, seed protein yield can be improved when temperatures of protein enrichment processes (e.g., pressing, expansion, or extrudation) are reduced by at least 1%, 5%, 10%, or 20% in comparison to processes where a control seed lot is processed under reduced temperatures. In certain embodiments, seed protein yield can be improved when temperatures of protein enrichment processes (e.g., pressing, expansion, or extrudation) are reduced by about 1%, 2%, or 5% to about 10% 20%, 30%, 40%, or 50% in comparison to processes where a control seed lot is processed under the same reduced temperatures. In certain embodiments, seed protein yield can be improved when energy inputs into an oil extraction process are reduced by at least 1%, 5%, 10%, or 20% in comparison to processes where a control seed lot is processed under the same reduced temperatures. In certain embodiments, seed protein yield can be improved when energy inputs into an oil extraction process are reduced by about 1%, 2%, or 5% to about 10%, 20%, 30%, 40%, or 50% in comparison to processes where a control seed lot is processed under the same reduced energy input conditions.

Seed, seed lots, food ingredients, and feed ingredients with increased oil content are provided as are methods for obtaining and using such seed, seed lots, food ingredients, and feed ingredients. In certain embodiments, the oil content of the seed, seed lots, food ingredients, and feed ingredients is increased by at least about 0.5%, 1%, or 2% per gram dry or wet weight of the in comparison to oil content of a control seed, seed lot, food ingredient, or feed ingredient. In certain embodiments, oil content of the seed, seed lots, food ingredients, and feed ingredients is increased by about 0.5%, 1%, or 2% to about 3%, 4%, 5%, 6%, 7%, 8%, 9%, or 10% per gram dry or wet weight of seed, seed lots, food ingredients, and feed ingredients in comparison to oil content of a control seed, seed lot, food ingredient, or feed ingredient. Controls include seed or seed lots harvested from mature control plants grown from an untreated control seed or untreated control plant as well as food and feed ingredients obtained from these control seed or seed lots.

Seed, seed lots, food ingredients, and feed ingredients with increased protein content are provided as are methods for obtaining and using such seed, seed lots, food ingredients, and feed ingredients. In certain embodiments, protein content is increased by at least about 0.5%, 1%, or 2% per gram dry or wet weight of the seed, seed lots, food ingredients, and feed ingredients in comparison to protein content of a control seed, seed lot, food ingredient, or feed ingredient. In certain embodiments, protein content of the seed, seed lots, food ingredients, and feed ingredients is increased by about 0.5% to about 1%, 2%, 3%, 4%, 5%, 6%, 7%, 8%, 9%, or 10% per gram dry or wet weight of the seed, seed lots, food ingredients, and feed ingredients in comparison to protein content of a control seed, seed lot, food ingredient, or feed ingredient. Controls include seed or seed lots harvested from mature control plants grown from an untreated control seed or untreated control plant as well as food and feed ingredients obtained from these control seed or seed lots.

Seed, seed lots, food ingredients, and feed ingredients with both increased oil and protein content are also provided as are methods for obtaining and using such seed, seed lots, food ingredients, and feed ingredients. In certain embodiments, protein content is increased by at least about 0.5%, 1%, or 2% per gram dry or wet weight of the seed, seed lots, food ingredients, and feed ingredients in comparison to protein content of a control seed, seed lot, food ingredient, or feed ingredient and oil content is increased by at least about 0.5%, 1%, or 2% per gram dry or wet weight of the in comparison to oil content of a control seed, seed lot, food ingredient, or feed ingredient. In certain embodiments, protein content is increased by about 0.5% to about 1%, 2%, 3%, 4%, 5%, 6%, 7%, 8%, 9%, or 10% per gram dry or wet weight of the seed, seed lots, food ingredients, and feed ingredients in comparison to protein content of a control seed, seed lot, food ingredient, or feed ingredient and oil content is increased by about 0.5%, 1%, or 2% to about 3%, 4%, 5%, 6%, 7%, 8%, 9%, or 10% per gram dry or wet weight of the seed, seed lots, food ingredients, and feed ingredients in comparison to oil content of a control seed, seed lot, food ingredient, or feed ingredient. Controls include seed or seed lots harvested from mature control plants grown from an untreated control seed or untreated control plant as well as food and feed ingredients obtained from these control seed or seed lots.

Plants, plant parts, food ingredients, and feed ingredients having increased levels of at least one mineral nutrient and/or at least one vitamin in comparison to a control plant, plant part, or feed ingredient are provided, as are methods for obtaining and using such plants, plant parts, food ingredients, and feed ingredients. In certain embodiments, the content of at least one mineral nutrient and/or at least one vitamin in the plants, plant parts, food ingredients, and feed ingredients is increased by at least about 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% per gram dry or wet weight in comparison to the content of the at least one mineral nutrient and/or at least one vitamin in a control plant, plant part, food ingredient, or feed ingredient. In other embodiments, the content of at least one mineral nutrient and/or at least one vitamin in the plants, plant parts, food ingredients, and feed ingredients is increased by more than 30%, including 35%, 40%, 45%, 50% or greater than 50% in comparison to the content of the at least one mineral nutrient and/or at least one vitamin in a control plant, plant part, food ingredient, or feed ingredient. In some embodiments, the content of more than one mineral nutrient and/or more than one vitamin is increased in a plant, plant part, food ingredient, and feed ingredient, and percent increases can vary for each of the mineral nutrients and/or vitamins, with each increased mineral nutrient and vitamin being increased by at least about 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% or more per gram dry or wet weight. Controls include plants or plant parts harvested from control plants grown from an untreated control seed or untreated control plant as well as food and feed ingredients obtained from these control plants or plant parts.

The mineral nutrient, vitamin, crude fat, oil, and/or protein content of whole seed, protein-enriched fractions, oil-enriched fractions, food ingredients, feed ingredients, and the like obtained from seed lots comprising at least 50%, 70%, 80%, 90%, or 95% of the seeds harvested from mature plants grown from seeds and/or plants treated with an effective amount of a Methylobacterium strain or strains can be determined by a variety of different techniques or combinations of techniques. Crude fat can be determined by methods comprising diethyl ether extraction of a dried sample, evaporation of the diethyl ether, and weighing the dried material recovered from the diethyl ether extract (Theix et al., 2003). Examples of crude fat determination methods include AOAC (formerly Association of Official Analytical Chemists) 920.39 and AACC (formerly American Association of Cereal Chemists) 30-20. Oil content can be determined by methods comprising nuclear magnetic resonance (NMR), near infrared spectroscopy (NIRS), gas-chromatography-mass spectroscopy (GC-MS), gas-chromatography-flame ionization detection (GC-FID), thin layer chromatography-flame ionization detection, liquid chromatography (LC)-mass spectroscopy (MS), liquid chromatography (LC)-electrospray ionization (EI)-mass spectroscopy (MS), or liquid chromatography (LC)-electrospray ionization (EI)-tandem mass spectroscopy (MS). Methods of determining oil content of seeds in general and single seeds by NIRS disclosed in U.S. Patent Appl. Pub. US20190003931, incorporated herein by reference in its entirety, can be adapted for use in the methods disclosed herein. Protein content can be determined by methods comprising nuclear magnetic resonance (NMR), near infrared spectroscopy (NIRS), a colorimetric assay, liquid chromatography (LC)-mass spectroscopy (MS), or matrix-assisted laser desorption/ionization time-of-flight mass spectrometry (MALDI-TOF). Assays for protein content can include colorimetric assays such as Lowry (Hartree, E. F., 1972) and Bradford (Bradford, 1976) assays. Protein content can also be determined indirectly by amino acid analysis or nitrogen determination (Mohre, et al., 2018). Nitrogen-based protein content determination methods include Dumas (Dumas, 1831) and Kjeldahl (Kjeldahl, 1883) methods. Nitrate and nitrite nitrogen content determination methods include Cadmium Reduction and Colorimetric analysis by Flow Injection system (Lachat); AOAC 968.07. Mineral Digestion can be accomplished by Open Vessel Microwave SW846-3051A (AOAC 991-10D(e)). Mineral analysis can be conducted by Inductively Coupled Argon Plasma (ICAP); AOAC 985.01. Mineral nutrients, vitamins, crude fat, oil, and protein content of seeds and various foods, feeds, feed ingredients, and food ingredients can also be determined by standard methods set forth by the AACC, AOAC in Official Methods of Analysis of AOAC INTERNATIONAL, 21st Edition (2019) and in the Codex Alimentarius of International Food Standards set forth by the Food and Agriculture Organization of the United Nations (FAO) or WHO (CXS 234-19991, Adopted in 1999).

### EXAMPLES

### Example 1. Effects of Methylobacterium Strain ISO10 (NRRL B-50938) Treatment of Soybean on Harvested Soybean Quality

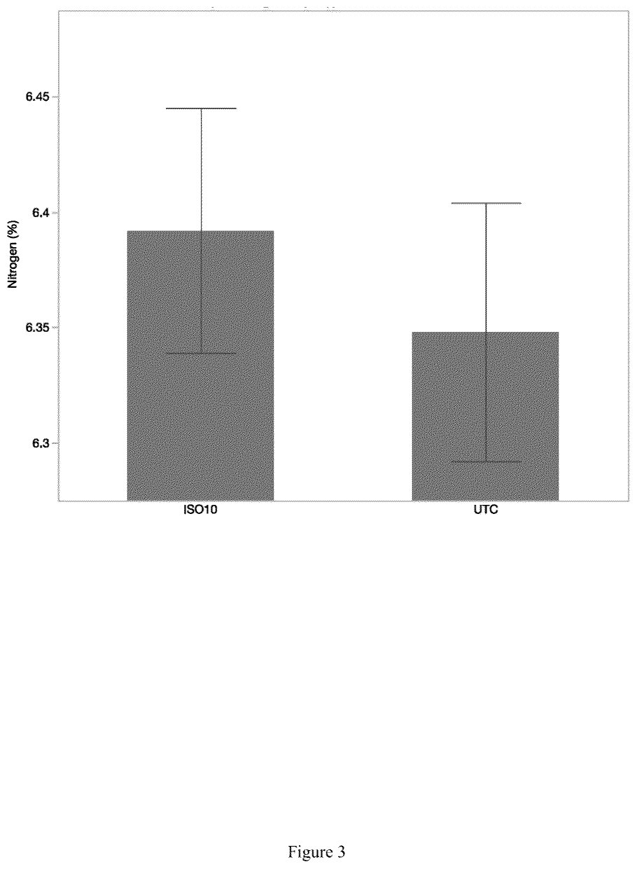

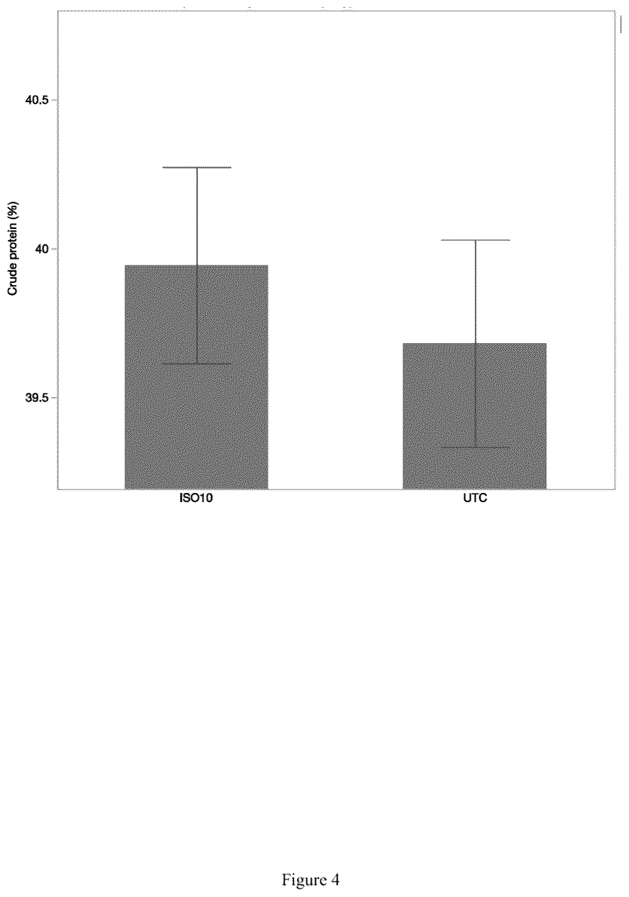

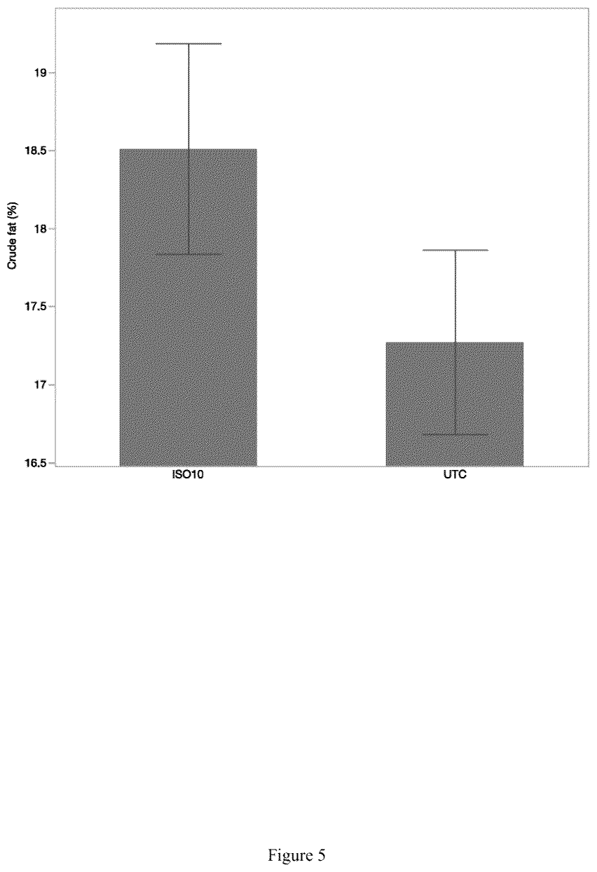

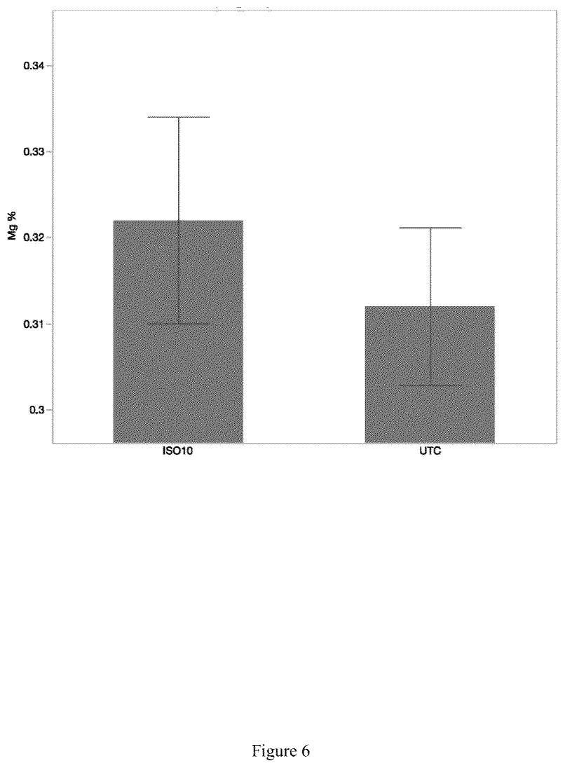

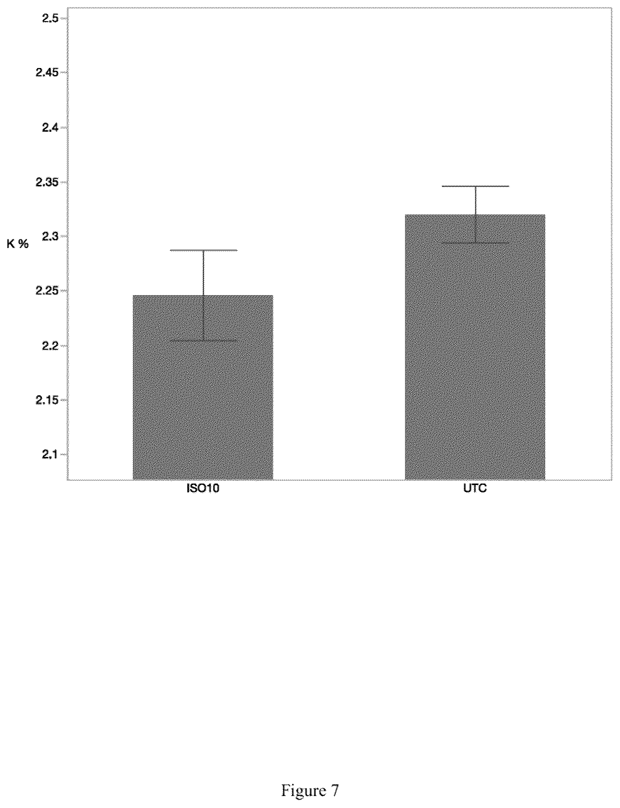

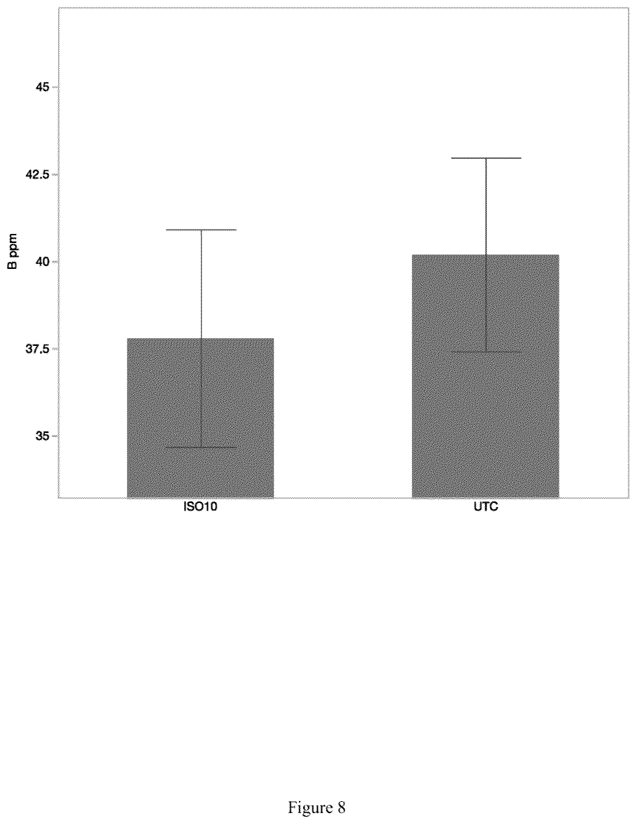

Soybean seeds (treated with standard seed applied fungicides and insecticides) were over-treated with Methylobacterium strain ISO10 at a rate of 10⁶ CFU per seed (NRRL B-50938) and grown at five distinct field locations in the Midwestern United States in the summer of 2018 in parallel with untreated control soybean plants. Locations were Cedar Falls, IA, Miami, MO, Dana, IA, Whitewater, WI and Aurora, NE. Seed was harvested from the treated and untreated control (UTC) soybean plants and assayed for micro- and macronutrient compositions. The results of the experiments are shown in FIG. 3 (percentage (%) nitrogen), FIG. 4 percentage (%) crude protein), FIG. 5 (percentage (%) crude fat), FIG. 6 (percentage (%) Magnesium), FIG. 7 percentage (%) Potassium), and FIG. 8 (percentage (%) Boron). Welch's two-tailed t-test with Location as a random effect was used for all statistical contrasts. Grain was combined for all ISO10 and UTC plots at each of five (5) locations, giving a sample size of 5 observations per treatment for every variable. Nutrient and crude fat/protein contents were measured as the percentage of dry weight of the sample.

Across the full panel of macro- and micronutrients tested and across crude protein & fat, ISO10 significantly altered the grain nutrient and quality profiles relative to the Untreated Control (UTC) (Table 2). Importantly, crude fat (determined by AACC\30-20) increased by 7.2% (p<0.0001) with ISO10 treatment. Crude protein and grain nitrogen contents (determined by AOAC (968.06) were higher with ISO10 treatment (0.7% increase for both, at significance levels of p=0.055 and 0.042, respectively). Grain magnesium content (determined by AOAC 975.03B(b)/AOAC 985.01) also increased significantly with ISO10 treatment (3.1% increase at p=0.0021). ISO10 treatment resulted in a significantly lower grain potassium content (3.3% decrease at p=0.0098) and marginally lower grain boron content (6.3% decrease at p=0.07) (as determined by AOAC 975.03B(b)/AOAC 985.01). Other macro- and micronutrients did not differ significantly between the ISO 10 treatment and the UTC.

TABLE 2
Mean nutrient contents on a dry weight basis, percent
difference of ISO10 from UTC, and contrast p-values.

| | | | | % dif- | Con- |
|---|---|---|---|---|---|
| Nutrient | | ISO10 | UTC | ference | trast |
| type | Nutrient (units) | value | value | from UTC | p-value |
| Macro- | Nitrogen (%) | 6.392 | 6.348 | +0.7% | [symbol] |
| nutrient | Phosphorus (%) | 0.624 | 0.644 | −3.2% | 0.17 |
| | Potassium (%) | 2.246 | 2.320 | −3.3% | [symbol] |
| | Calcium (%) | 0.390 | 0.356 | +8.7% | 0.40 |
| | Magnesium (%) | 0.322 | 0.312 | +3.1% | [symbol] |
| | Sulfur (%) | 0.342 | 0.338 | +1.2% | 0.43 |
| Micro- | Zinc (ppm) | 40 | 40 | 0 | 1.00 |
| nutrient | Manganese (ppm) | 36.4 | 36.4 | 0 | 1.00 |
| | Iron (ppm) | 79.6 | 78.0 | +2.0% | 0.57 |
| | Copper (ppm) | 13.6 | 13.2 | +2.9% | 0.43 |
| | Boron (ppm) | 37.8 | 40.2 | −6.3% | 0.071 |
| | Crude protein (%) | 39.944 | 39.680 | +0.7% | 0.055 |
| | Crude fat (%) | 18.512 | 17.272 | +7.2% | [symbol] |

Bold italics indicate p < 0.05.

### Example 2. Effects of Methylobacterium Strain ISO10 (NRRL B-50938) Treatment of Soybean Seeds on Nutrient Content of Vegetative Tissue

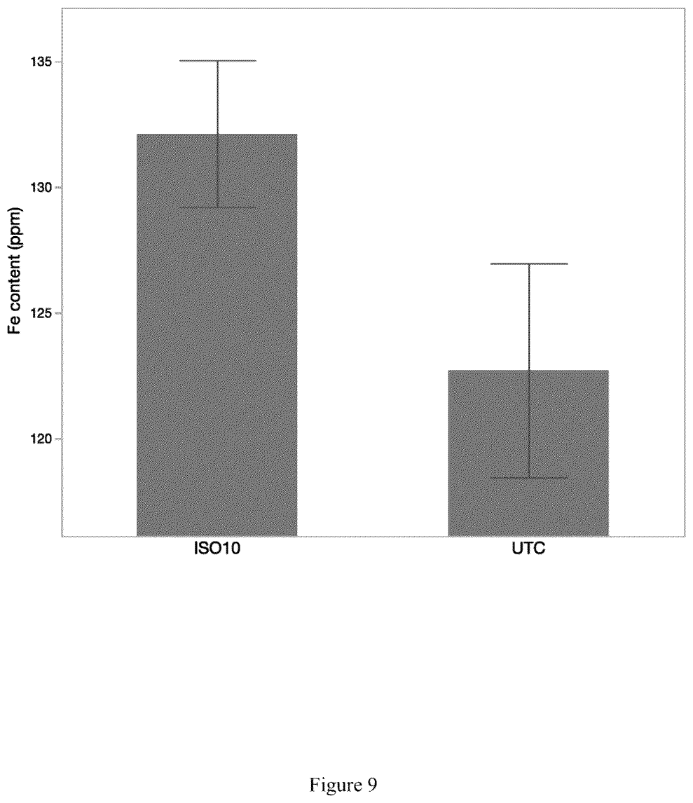

Soybean seeds were treated with Methylobacterium strain ISO10 at a rate of 10⁶ CFU per seed and grown in sterilized soil (30 seeds per flat) in a greenhouse in parallel with untreated soybean seeds. At 22 days after planting (V1 growth stage), 25 plants per flat were chosen randomly and the youngest fully expanded trifoliate leaf was cut off of each plant. The trifoliates were incubated in sample bags at 45° C. for 4 days to dry and analyzed (Table 3) for macronutrient and micronutrient content. A single-tailed unequal variances (Welch's) t-test was used to determine whether treatment with ISO 10 resulted in a significant increase in nutrient content. Iron showed a significant enhancement relative to the UTC. Mean foliar iron content was 7.7% greater following treatment with ISO10, a statistically significant difference (p 0.0403). Results are shown in FIG. 9.

TABLE 3
Mean nutrient contents on a dry weight basis, percent
difference of ISO10 from UTC, and contrast p-values.

| | | | | % dif- | Con- |
|---|---|---|---|---|---|
| Nutrient | | ISO10 | UTC | ference | trast |
| type | Nutrient (units) | value | value | from UTC | p-value |
| Macro- | Nitrogen (%) | 3.034 | 3.064 | −1.0% | 0.71 |
| nutrient | NO3—N (%) | 0.0038 | 0.0050 | −24.0% | 0.92 |
| | Phosphorus (%) | 0.151 | 0.154 | −1.9% | 0.74 |
| | Potassium (%) | 2.573 | 2.577 | −0.2% | 0.56 |
| | Calcium (%) | 1.615 | 1.606 | +0.6% | 0.41 |
| | Magnesium (%) | 0.522 | 0.517 | +1.0% | 0.39 |
| | Sulfur (%) | 0.182 | 0.180 | +1.1% | 0.31 |
| Micro- | Zinc (ppm) | 21.8 | 21.6 | +0.9% | 0.38 |
| nutrient | Manganese (ppm) | 98.6 | 95.7 | +3.0% | 0.17 |
| | Iron (ppm) | 132.1 | 122.7 | +7.7% | [symbol] |
| | Copper (ppm) | 5.4 | 5.5 | −1.8% | 0.63 |
| | Boron (ppm) | 42.7 | 43.5 | −1.8% | 0.76 |

Bold italics indicate p < 0.05.

### Example 3. Effects of Methylobacterium Strain ISO20 (NRRL B-67743) Treatment of Corn on Nutrient Content of Vegetative Tissue

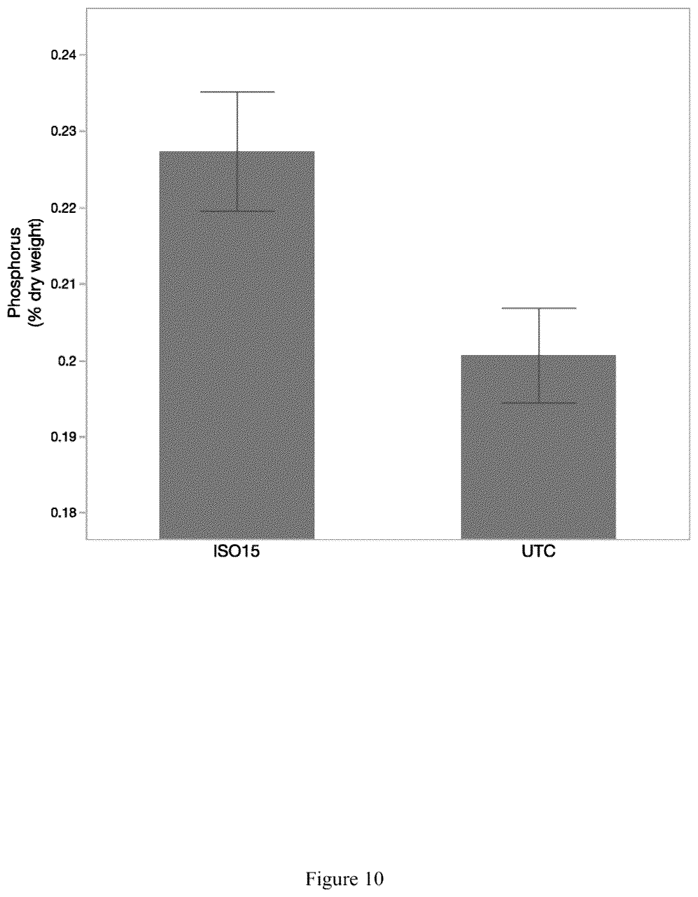

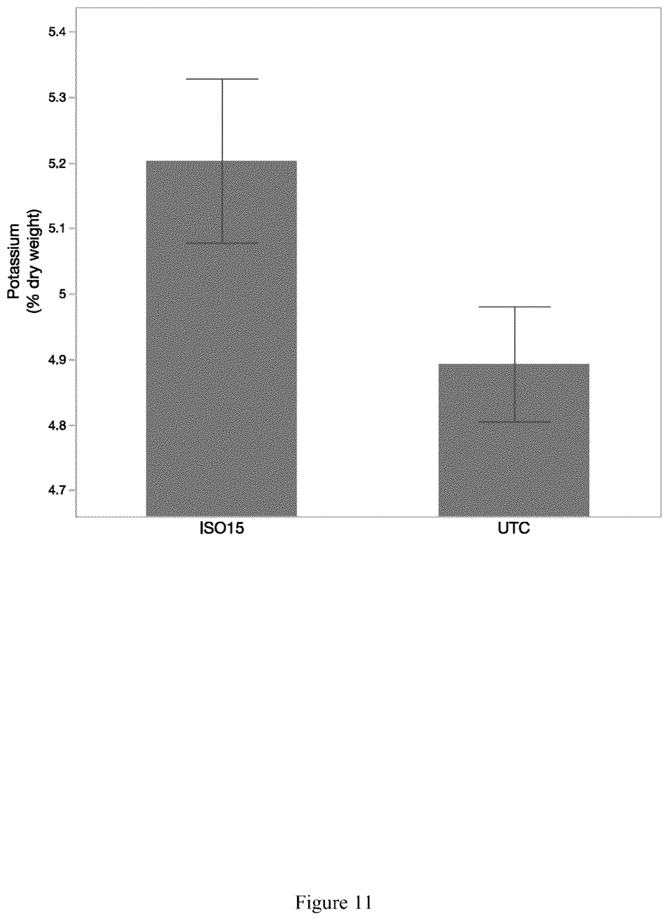

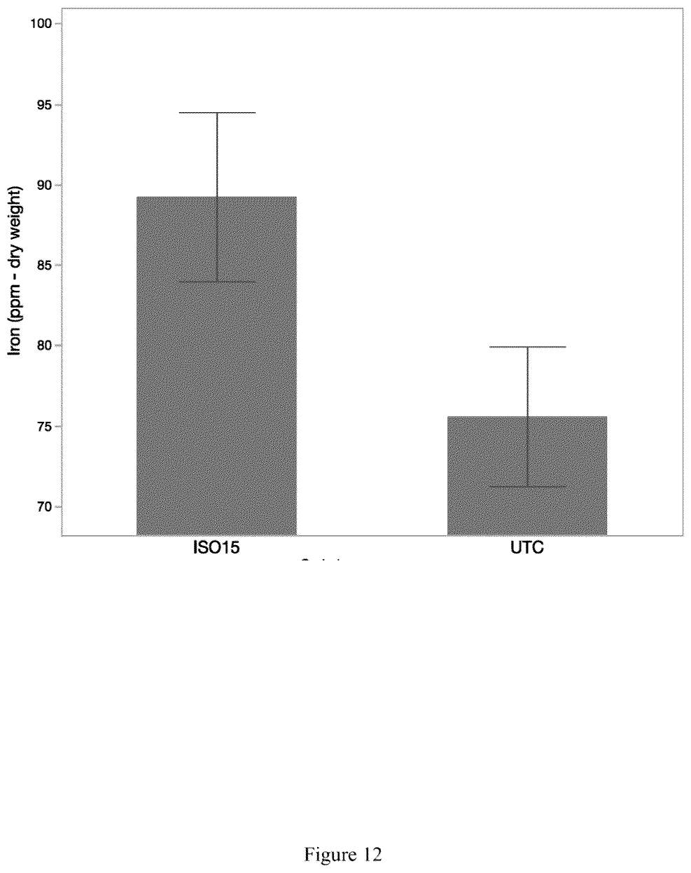

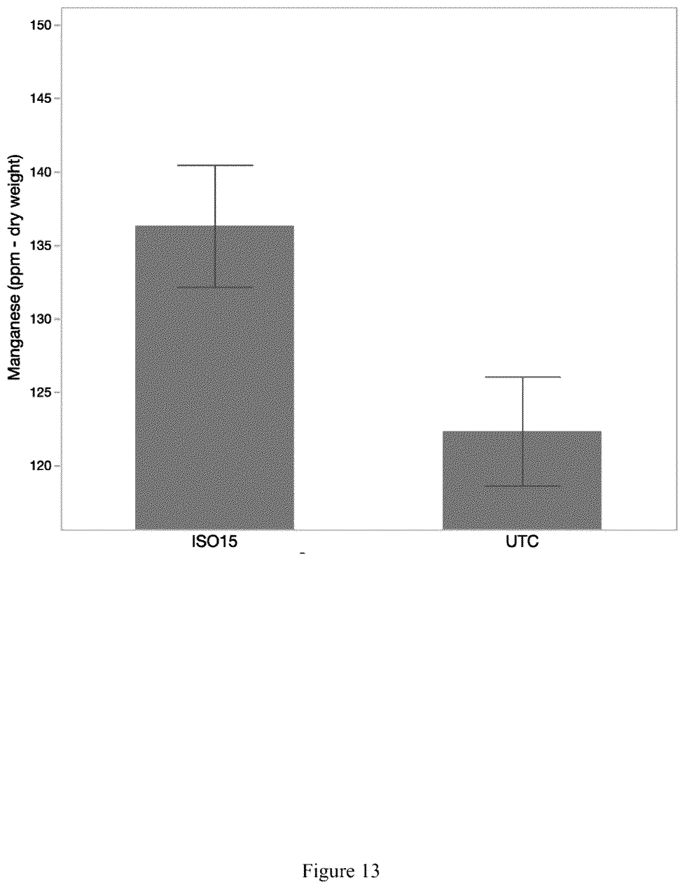

Corn seeds were treated with Methylobacterium strain ISO20 at a rate of 10⁶ CFU per seed and grown in sterilized soil (30 seeds per flat) in a greenhouse in parallel with untreated corn seeds. At 22 days after planting (V2 growth stage), 15 or more plants per flat were chosen randomly and shoots were collected by cutting one inch above the soil line. The shoots were incubated in sample bags at 45° C. for 4 days to dry and analyzed for macronutrient and micronutrient content. A single-tailed unequal variances (Welch's) t-test was used to analyze the data to determine whether treatment with ISO 15 resulted in a significant increase in nutrient content. Methylobacterium ISO 15 significantly enhanced foliar content of four nutrients: phosphorus (P), potassium (K), iron (Fe) and manganese (Mn). Of the 12 nutrients tested, 9 were elevated over the UTC by treatment with ISO 15. Results are shown in Table 4 and FIGS. 10-13.

TABLE 4
Mean nutrient contents on a dry weight basis, percent
difference of ISO20 from UTC, and contrast p-values.

| | | | % | |
|---|---|---|---|---|
| | ISO20 | UTC | difference | Contrast |
| Nutrient (units) | value | value | from UTC | p-value |
| Nitrogen (%) | 1.466 | 1.465 | +0.1% | 0.49 |
| NO3—N (%) | 0.0024 | 0.0021 | +14.3% | 0.17 |
| Phosphorus (%) | 0.227 | 0.201 | +12.9% | [symbol] |
| Potassium (%) | 5.20 | 4.90 | +6.1% | [symbol] |
| Calcium (%) | 0.839 | 0.815 | +2.9% | 0.28 |
| Magnesium (%) | 0.254 | 0.251 | +1.2% | 0.38 |
| Sulfur (%) | 0.216 | 0.226 | −4.4% | 0.80 |
| Zinc (ppm) | 22.8 | 21.6 | +5.6% | 0.15 |
| Manganese (ppm) | 136.3 | 122.3 | +11.4% | [symbol] |
| Iron (ppm) | 89.3 | 75.6 | +18.1% | [symbol] |
| Copper (ppm) | 6.13 | 6.33 | −3.2% | 0.73 |
| Boron (ppm) | 5.47 | 5.53 | −1.1% | 0.57 |

Bold italics indicate p < 0.05.

### Example 4 Detection or Identification of Methylobacterium Strains, Variants and Derivatives

Assays are disclosed for detection or identification of specific Methylobacterium strains and closely related derivatives. Genomic DNA fragments unique to a Methylobacterium strain are identified and qPCR Locked Nucleic Acid (LNA) based assays are developed.

Genomic DNA sequences of Methylobacterium strains are compared by BLAST analysis of approximately 300 bp fragments using a sliding window of from 1-25 nucleotides to whole genome sequences of over 1000 public and proprietary Methylobacterium isolates. Genomic DNA fragments are identified that have weak BLAST alignments, indicative of approximately 60-95% identity over the entire fragment, to corresponding fragments of a Methylobacterium of interest. Fragments from the NLS0109 genome corresponding to the identified weak alignment regions were selected for assay development and are provided as SEQ ID NOS:1-3.

TABLE 5
Target Fragment Sequences of NLS0109

| | SEQ | |
|---|---|---|
| | ID | |
| Fragment | NO | Sequence |
| ref1_135566 | 1 | ACGGTCACCCCACGGACTGGGCGAGTACCTCACCGGTGTTCTA |
| | | TCATAACGCCGAGTTAGTTTTCGACCGTCCCTTATGCGATGTA |
| | | CCACCGGTGTCGGCAGCCGATTTCGTCCCACCGGGAGCTGGCG |
| | | TTCCGGTTCAGACCACCATCATCGGTCACGATGTCTGGATTGG |
| | | ACACGGGGCCTTCATCTCCCCCGGCGTGACTATAGGAAACGGC |
| | | GCGATCGTCGGGGCCCAGGCGGTCGTCACAAGAGATGTCCCA |
| | | CCCTATGCGGTAGTTGCTGGCGTCCCCGCGACCGTACGACGAT |
| ref1_135772 | 2 | CCAATAAAAGCGTTGGCCGCCTGGGCAACCCGATCCGAGCCT |
| | | AAGACTCAAAGCGCAAGCGAACACTTGGTAGAGACAGCCCGC |
| | | CGACTACGGCGTTCCAGCACTCTCCGGCTTTGATCGGATAGGC |
| | | ATTGGTCAAGGTGCCGGTGGTGATGACCTCGCCCGCCGCAAGC |
| | | GGCGAATTACTCGGATCAGCGGCCAGCACCTCGACCAAGTGT |
| | | CGGAGCGCGACCAAAGGGCCACGTTCGAGGACGTTTGAGGCG |
| | | CGACCAGTCTCGATAGTCTCATCGTCGCGGCGAAGCTGCACCT |
| | | CGA |
| ref1_169470 | 3 | CGATGGCACCGACCTGCCATGCCTCTGCCGTCCGCGCCAGAAT |
| | | GGTAAAGAGGACGAAGGGGGTAAGGATCGTCGCTGCAGTGTT |
| | | GAGCAGCGACCAGAGAAGGGGGCCGAACATCGGCATCAAACC |
| | | TCGATTGCCACTCGGACGCGAAGCGCGTCTTGAAGGAGGGAT |
| | | GGAAGCGAAACGGCCGCAGAGTAACCGCCGACGAAAGATTGC |
| | | ACCCCTCATCGAGCAGGATCGGAGGTGAAGGCAAGCGTGGGT |
| | | TATTGGTAAGTGCAAAAAATATAATGGTAGCGTCAGATCTAGC |
| | | GTTC |

Regions in SEQ ID NOS: 1-3 where corresponding regions in other Methylobacterium strains were identified as having one or more nucleotide mismatches from the NLS0109 sequence were selected, and qPCR primers designed using Primer3 software (Untergasser et al. (2012), Koressaar et al. (2007)) to flank the mismatch regions, have a melting temperature (Tm) in the range of 55-60 degrees, and to generate a PCR DNA fragment of approximately 100 bp. The probe sequence was designed with a 5′ FAM reporter dye, a 3′ Iowa Black FQ quencher, and contains one to six LNA bases (Integrated DNA Technologies, Coralville, Iowa). At least 1 of the LNA bases is in the position of a mismatch, while the other LNA bases are used to raise the Tm. The Tm of the probe sequence is targeted to be 10 degrees above the Tm of the primers. Primer and probe sequences for detection of specific detection of NLS0109 are provided as SEQ ID NOS: 4-12 in Table 6. Each of the probes contains a 5′ FAM reporter dye and a 3′ Iowa Black FQ quencher.

TABLE 6
Primer and Probe Sequences for Specific
Detection of NLS0109

| | SEQ | |
|---|---|---|
| | ID | |
| Primer/Probe | NO | Sequence* |
| NLS0109_ref1_135566_forward | 4 | CCTCACCGGTGTTCTA |
| | | TCATAAC |
| NLS0109_ref1_135566_reverse | 5 | CCGATGATGGTGGTCT |
| | | GAAC |
| NLS0109_ref1_135566_probe | 6 | CGTCCCTTATGCGATG |
| | | TACCA |
| NLS0109_ref1_135772_forward | 7 | GATCCGAGCCTAAGAC |
| | | TCAAAG |
| NLS0109_ref1_135772_reverse | 8 | GACCAATGCCTATCCG |
| | | ATCAA |
| NLS0109_ref1_135772_probe | 9 | AACACTTGGTAGAGAC |
| | | AGCC |
| NLS0109_ref1_169470_forward | 10 | AAGGAGGGATGGAAGC |
| | | GAAAC |
| NLS0109_ref1_169470_reverse | 11 | ATAACCCACGCTTGCC |
| | | TTC |
| NLS0109_ref1_169470_probe | 12 | CGCAGAGTAACCGCCG |
| | | ACGAA |

*Bold and underlined letters represent the position of an LNA base

Use of Primer/Probe Sets on Isolated DNA to Detect NLS0109 and Distinguish from Related Methylobacterium Isolates

Each 10 ul qPCR reaction contains 5 ul of Quantabio PerfeCTa qPCR ToughMix 2× Mastermix, Low ROX from VWR, 0.5 ul of 10 uM forward primer, 0.5 ul of 10 uM reverse primer, 1 ul of 2.5 uM probe, 1 ul nuclease free water and 2 ul of DNA template. Approximately 1 ng of DNA template is used per reaction. The reaction is conducted in a ThermoFisher QuantStudio™ 6 Flex Real-Time PCR System with the following program: 95° C. for 3 min, then 40 cycles of 95° C. for 15 sec and 60° C. for 1 min. The analysis software on the PCR instrument calculates a threshold and Ct value for each sample. Each sample was run in triplicate on the same qPCR plate. A positive result is indicated where the delta Ct between positive and negative controls is at least 5.

Use of the three primer/probe sets to distinguish NLS0109 from closely related isolates by analysis of isolated DNA is shown in Table 7 below. The similarity score shown for the related isolates takes into account both the average nucleotide identity and the alignment fraction between the isolates and NLS0109. One of the tested strains, NLS0730, was used as an additional positive control. NLS0730 is a clonal isolate of NLS109 which was obtained from a culture of NLS0109, which was confirmed by full genome sequencing as identical to NLS0109, and which scored positive in all three reactions. The similarity score of greater than 1.000 for this strain is likely the result of a slightly different assembly of the genome for this isolate compared to NLS0109. The delta Ct of approximately 15 or more between the NLS0109 and NLS0730 isolates and the water only control is consistent with the sequence confirmation of the identity of these isolates. Analysis of other isolates that are less closely related to NLS0109 results in delta Ct values similar to those for the water only control.

TABLE 7

| | Similarity score | Average Ct Value | | |
|---|---|---|---|---|
| NLS# | to NLS0109 | Ref1_135566 | Ref1_135772 | Ref1_169470 |
| NLS0730 | 1.005 | 21.08 | 21.31 | 20.35 |
| NLS0109 | 1 | 21.97 | 22.62 | 22.08 |
| NLS0731 | 0.181 | No Ct | 37.85 | >37.91 |
| NLS0644 | 0.87 | >36.8 | >38.31 | No Ct |
| NLS0700 | 0.88 | >38.36 | >38.36 | >38.44 |
| NLS0710 | 0.894 | No Ct | >37.47 | >38.13 |
| NLS0834 | 0.852 | 37.81 | No Ct | 37.97 |
| NLS0939 | 0.862 | 37.94 | 38.37 | >38.35 |
| NLS0947 | 0.807 | 38.44 | No Ct | No Ct |
| NLS1015 | 0.894 | 38.77 | No Ct | >37.91 |
| NLS1217 | 0.872 | 37.64 | 37.20 | 37.96 |
| H2O only | | >38.14 | >35.92 | >37.12 |

### Use of Primer/Probes for Detection of NLS109 on Treated Plant Materials.

For detection of NLS0109 foliar spray treatment on corn: Untreated corn seeds were planted in field soil in the growth chamber and watered with non-fertilized R.O. water. After plants germinated and grew for approximately 3 weeks, they were transferred to the greenhouse. At V5 stage, plants were divided into 3 groups for treatment: foliar spray of NLS0109, mock foliar spray, and untreated. Plants receiving the foliar spray of NLS0109 were treated with 10× glycerol stock at the rate of 71.4 ul per plant using Solo sprayers. This converts to the rate of 10 L/acre in the field. Mock treated plants were sprayed with 71.4 ul water/plant. Untreated plants received no foliar spray treatment. Leaves were harvested two weeks after foliar spray treatment into sterile tubes and DNA from bacteria on the harvested leaves is isolated as described above. Each experiment was grown at least 2 times. As shown in Table 8, NLS0109 is detected on leaves harvested from corn plants treated by a foliar spray application of the Methylobacterium strains using all 3 primer probe sets, as demonstrated by delta Ct values of approximately 10 between the sample and the negative controls.

TABLE 8

| | Average Ct Value | | |
|---|---|---|---|
| Treatment | Ref1_135566 | Ref1_135772 | Ref1_169470 |
| Control (no application) | 32.43 | 32.10 | 31.55 |
| Control (mock application) | 35.54 | 35.34 | 34.80 |
| NLS0109 (10 L/acre equivalent) | 23.36 | 22.88 | 22.66 |

The above results demonstrate the use of genome specific primers and probes to detect Methylobacterium strain NLS0109 on various plant tissues following treatment with the strains and provide methods to distinguish NLS0109 from closely related isolates. Similar methods are developed for additional Methylobacterium strains, NLS0017, NLS0807, NLS0662 and NLS0648 using target sequence fragments and primer/probe pairs as shown in the Tables below.

TABLE 9
Target Fragment Sequences of NLS0017

| | SEQ | |
|---|---|---|
| | ID | |
| Fragment | NO | Sequence |
| ref4_930 | 13 | GCAAAACGACCTAATAGTTCTACAGCGGCATGC |
| | | GCCAAGTCAGCGCGGTGAACAGTATACCTGGGA |
| | | GCAACTTGTCCTCCGAAACCCACATAAAACAAA |
| | | TTACTCCTGGCAGTGCCCAGTCCATCAAAATCG |
| | | AATACAATATTTCTCGAGGAGGCATCTGTAATA |
| | | GCCTGCCAAAGCAACAAAGCTATGGCGCCGTTA |
| | | TGACTTTCATTGCTTCTGGTAGACATAAAATAA |
| | | TATGCCGATTTGTGATCCCAAATGTAGAATATT |
| | | GCCGCATCAATTGCGCCAAGTTTATTTCGGATC |
| | | GAT |
| ref1_142021 | 14 | GGCGCCAACGGTATGATCGCATGATTTTCCTGC |
| | | GGCATAGCTTGCGGGAATGGCGTATTTGGCGCT |
| | | CTCCTCAGGAATTTCTAAGGGCATACGCAGGAA |
| | | CTCTACAGCACTTTTACTGGTATTTTGTAGTGA |
| | | CAGCGGAGGAGGCTGGTGCTCAAGGTAATCGTG |
| | | ATGAAGTGATCCGGGCCATTCGGGGCGCGTTTC |
| | | TAGTCTTTCCAATCCGCGCCCTGTACCACGTAT |
| | | TACGCCGGACCGGTCTGCGCCGCGCCGCCCTCT |
| | | TGACCGCCCTAAATGTCTAAGAGCGTCTAACAA |
| | | AGC |
| ref1_142636 | 15 | GACGATATCGCTCATCTTCACTGCATTGAAGCT |
| | | GGTGCCGTACTGCATAGGGATGAAAAAGTGATG |
| | | CGGATAGACGGCTGACGGGAAAGCGCCTGGTCG |
| | | ATCGAAGACTTTGCTGACGAGGTTGTGGTAGCC |
| | | CCGGATATAGGCATCGAAGGCCGGGACGTTGAT |
| | | CCCATCCTTTGCCTTATCTTGACTGGCGTCGTC |
| | | GCGTGCCGTCAGAACGGGCACGTCGCAGGTCAT |
| | | CGAGGCCAGCACCTTGCGGAACACCTGCGTTCC |
| | | GCCGTTGGGATTATCGACGGCGAACGCGGTGGC |
| | | CGC |

TABLE 10
Primer and Probe Sequences for Specific
Detection of NLS0017

| | SEQ | |
|---|---|---|
| | ID | |
| Primer/Probe | NO | Sequence* |
| NLS0017_ref4_930_forward | 16 | GTCCTCCGAAACCCACAT |
| | | AAA |
| NLS0017_ref4_930_reverse | 17 | CTACCAGAAGCAATGAAA |
| | | GTCAT |
| NLS0017_ref4_930_probe | 18 | TCTGTAATAGCCTGCCAA |
| | | AGCA |
| NLS0017_ref1_142021_forward | 19 | GGCTGGTGCTCAAGGTAA |
| | | T |
| NLS0017_ref1_142021_reverse | 20 | ACATTTAGGGCGGTCAAG |
| | | AG |
| NLS0017_ref1_142021_probe | 21 | ATGAAGTGATCCGGGCCA |
| | | T |
| NLS0017_ref1_142636_forward | 22 | CCGTACTGCATAGGGATG |
| | | AAA |
| NLS0017_ref1_142636_reverse | 23 | TAAGGCAAAGGATGGGAT |
| | | CAA |
| NLS0017_ref1_142636_probe | 24 | TTGCTGACGAGGTTGTGG |
| | | TAG |

*Bold and underlined letters represent the position of an LNA base

TABLE 11
Target Fragment Sequences of NLS0807

| | SEQ | |
|---|---|---|
| | ID | |
| Fragment | NO | Sequence |
| ref1_458355 | 25 | CAACTATGTAGACCCGACGGTGCGATTTCACTTCGCAAAGCCG |
| | | CAGGGCAGCACCCTTGCGCTCAATGTTGACGCCAGCGTGATCT |
| | | ATACTATTACCGTCACGCACACGCAGGGCGGCGTACAGATTCA |
| | | TCGCGAGAGTAAGAACCACCATCAGACCATCACGCGCAGCGA |
| | | CCTGAGCAAGCAGTTCGGCGTTGGTGTGGCCGACCAGCTGAC |
| | | GCGCGATCAGGTCATGAAGGTGATCGAGTCGGCATTTCGCGA |
| | | CGCTACCCGCTAAGATCGGCGCCCACGAAACGCTACGAGACT |
| | | AGG |
| ref1_459688 | 26 | AGCCGGCATCTTGTTCAAGGCGCTCACCTCGACGCCGACGCTG |
| | | TAGGCGACTTGAGAGGGCGTCTCATATGAACGAAGCATCTTCG |
| | | CGTAGAGAACCTTCTTGTTCTCCTGCGTGATGTTCGCTTTGCAG |
| | | ACGTTGACTGCCGCCATGAACGCCGAAGCCTTGCGCGCTTCAT |
| | | CGTAATCGCCTGCGAAGGCGGGTAGTGAAAAGCTTAGTGCAA |
| | | TGGCAAACACAGCCGCCGAACGTCGCATGGTATCCGTCCCCG |
| | | ATTGACGGCAGTGCCGCCATATCTCGGCTTTAGCAGAGCTGAT |
| ref1_3158527 | 27 | AACCTGCGCCGGCCGAGGTTTCGCGAGCCGTCGCCACGGGCA |
| | | ACGCCTCGCCCGCGATGTGCAAAAAAGTCCCCGGCACTTCGCG |
| | | CCGTCGTCCGATCCACGACCGCGAATTTCTCAACGAGTACAAG |
| | | GTGCTTATGGGAGATCCGAGCGTCCGTCCCGGAGCCCGAGAC |
| | | CGCGCGGCCCGAGTAATAGGCGAAAAAGACTCCTACTCCTCG |
| | | GGCTTCTCGGGCCCCCTCAGCAACATCTACGCTTGCCGCCCAT |
| | | CACCCTGGCGGGAGATCAGCGACGAGACACAGGCCCACTTCG |
| | | CCC |

TABLE 12
Primer and Probe Sequences for Specific
Detection of NLS0807

| | SEQ | |
|---|---|---|
| | ID | |
| Primer/Probe | NO | Sequence* |
| NLS0807_ref1_458355_forward | 28 | TTGACGCCAGCGTGA |
| | | TCTATAC |
| NLS0807_ref1_458355_reverse | 29 | GTGATGGTCTGATGG |
| | | TGGTTCT |
| NLS0807_ref1_458355_probe | 30 | TATTACCGTCACGCA |
| | | CACG |
| NLS0807_ref1_459688_forward | 31 | CTTCGCGTAGAGAAC |
| | | CTTCTTGTT |
| NLS0807_ref1_459688_reverse | 32 | CTTCGCAGGCGATTA |
| | | CGATGAA |
| NLS0807_ref1_459688_probe | 33 | CGTGATGTTCGCTTT |
| | | GCAGA |
| NLS0807_ref1_3158527_forward | 34 | CCGCGAATTTCTCAA |
| | | CGAGTACA |
| NLS0807_ref1_3158527_reverse | 35 | GCCCGAGGAGTAGGA |
| | | GTCTTT |
| NLS0807_ref1_3158527_probe | 36 | AGGTGCTTATGGGAG |
| | | ATCCG |

*Bold and underlined letters represent the position of an LNA base

Use of the primer/probe sets to distinguish NLS0807 from closely related isolates by analysis of isolated DNA is shown in Table 13 below. The similarity score shown for the related isolates takes into account both the average nucleotide identity and the alignment fraction between the isolates and NLS0807. Two of the tested strains, NLS0821 and NLS0044, were used as additional positive controls since a similarity score of 1.00 indicates they are nearly identical to NLS0807. Consistently low Ct values from qPCR using NLS0807 as the DNA template and no detection in the water only control is consistent with the sequence confirmation of the identity of these isolates. Analysis of other isolates that are less closely related to NLS0807 results in no detection similar to those for the water only control.

TABLE 13

| | Similarity | | | |
|---|---|---|---|---|
| | to | Average Ct Value | | |
| NLS# | NLS0807 | ref1_459688 | ref1_3158527 | ref1_458355 |
| NLS0807 | 1.00 | 22.39 | 24.09 | 23.10 |
| NLS0821 | 1.00 | 22.49 | 24.04 | 22.96 |
| NLS0044 | 1.00 | 22.49 | 23.86 | 22.90 |
| Strain A | 0.95 | UDT | UDT | UDT |
| Strain B | 0.94 | UDT | UDT | UDT |
| Strain C | 0.93 | UDT | UDT | UDT |
| Strain D | 0.93 | UDT | UDT | UDT |
| water only (neg control) | — | UDT | UDT | UDT |

TABLE 14
Target Fragment Sequences of NLS0648

| | SEQ | |
|---|---|---|
| | ID | |
| Fragment | NO | Sequence |
| ref1_1185955 | 37 | AGTCATTGATCAAGCAACCCCTATTGAGTTGGATATCGAAGGA |
| | | TCAAGGTCGCGTCAATAGATGCATCTATCAGGCCAAATGTCGC |
| | | TTTTCAAGAATGGCTCTTTCGAAGCTATCTTTATAATCGCTCGC |
| | | CATTCTCTCATTACCAAAATCGACCTTAACTAGCTCGACATTG |
| | | ATGCGAGCAGCTCCGGCAAACGAGGAGAGATTGACCTTAAAG |
| | | GAATTGAACGCCTCAAGCAATTCAGACACATTACCAGGAGTG |
| | | CTATAGCAACAACCAGACCCATATCGGTCAATAACCTCTTTTA |
| ref1_3282585 | 38 | CGCAAAACGATTTATCACTGCCATCTTGTTGTTTGATAACCCTT |
| | | TTTTACCAGACGTTATGCTGGGCGAGAAAGAGGACTAGCAGA |
| | | TCGGAGCGGTATCGCGATTTTTCGGTAGTTCGCGCCTACAACA |
| | | GGATAAGATCCGATAGTGAAGCAACATGGCTGTTTTTTGATTT |
| | | GTAAGTCAGCAACTTAAGCAGCCAGCCTATCTGCCGTCGCAGA |
| | | CGCTTGAGGCATCGGGCAGCATCTTAGAAAAGGTGGCAGTAA |
| | | TTGCCACAGCGGAACGTAGCGGCACGGATAAGCACGCAGGGT |
| | | C |
| ref1_4194637 | 39 | CCCATCTGGACCCAATATCCCCTTCATCGACAATTCCCGAGTA |
| | | AGTGTGGGTTCGAGGATTTCGCGAAACAGCCTTGTTCGTTCCT |
| | | CCGGCCTTAAAATTGGCGTGCCGTCGGGAGATCGATAGGCATC |
| | | CCTTACCTGCCTTTCGACCGCCGGCACACGCGCGCCGGTCGTC |
| | | GTGTTCACGGCCACGGAATGGACGAAGGTGCGCCGCTCATTTC |
| | | GCTCGTTTGCCGTCTCCACCATCCAGGAGGCCAGCAGGACGGT |
| | | TTCGTCTCGACCGCCGGTCACACACACCGCAAGGGACTCAGG |

TABLE 15
Primer and Probe Sequences for Specific
Detection of NLS0648

| | SEQ | |
|---|---|---|
| | ID | |
| Primer/Probe | NO | Sequence* |
| NLS0648_ref1_1185955_forward | 40 | TCGCTCGCCATTCTC |
| | | TCATTAC |
| NLS0648_ref1_1185955_reverse | 41 | AGGTCAATCTCTCCT |
| | | CGTTTGC |
| NLS0648_ref1_1185955_probe | 42 | TCGACATTGATGCGA |
| | | GCA |
| NLS0648_ref1_3282585_forward | 43 | TTCGCGCCTACAACA |
| | | GGATAAG |
| NLS0648_ref1_3282585_reverse | 44 | CAGATAGGCTGGCTG |
| | | CTTAAGTT |
| NLS0648_ref1_3282585_probe | 45 | TCCGATAGTGAAGCA |
| | | ACA |
| NLS0648_ref1_4194637_forward | 46 | GAGTAAGTGTGGGTT |
| | | CGAGGATTT |
| NLS0648_ref1_4194637_reverse | 47 | AGGTAAGGGATGCCT |
| | | ATCGATCT |
| NLS0648_ref1_4194637_probe | 48 | CGGAGGAACGAACAA |
| | | GGC |

*Bold and underlined letters represent the position of an LNA base

TABLE 16
Target Fragment Sequences of NLS0662

| | SEQ | |
|---|---|---|
| | ID | |
| Fragment | NO | Sequence |
| NLS0662_ref1_4871392 | 49 | ACCTGCTAAAATCACGTCCTCTCAGATTGAAAAAT |
| | | CATTGAAGAAACGTGTCGAACGATTGCCGGGGATT |
| | | ATGACGTTAGATCAATTGAAAAATACAAGCTTTGA |
| | | AATTGAGTTACAGCCAAAAGATGCCCCGGATCCGG |
| | | ACCCATCAGACTTCGGTGGCTAGTTCGAGCCAAAC |
| | | TCGAACGTCGCCATGGCGCGCAAGTCGCAATACCA |
| | | TTTCACAGCGCAGCGGTTATTTCGTTGTACACTGTA |
| | | GCAATGCGTCGGCTTGCGCGCTTCCGCTGGCGATC |
| | | AAAGGTCCGCCGATTTACG |
| NLS0662_ref1_1266930 | 50 | TCCCGAACATACAATGGAGGAAGCGTGTGGTAGGC |
| | | CAATTTGTAACGAAATATGGCATCGGTCACGGCTC |
| | | TCTCAATAAATTCGATCTCAAGTCTTCTGAACGAG |
| | | CATGCCTCATCCTTATCCTGAGCGAACGCCTGCCA |
| | | GTTTGCAGTCATTCCAACATACATAGCCAAAAAGG |
| | | CGAGGTAGACCTTCATACGGGCACCTCAATCGTCC |
| | | CCATTCGTTCAAGCTCCTTCAAGATAACAGCCGCA |
| | | CCACATTGCTGAGATCGAAGATTCGGATCAAATAT |
| | | TCCATCAAATTTATACTTTC |
| NLS0662_ref1_17614 | 51 | GCATCCTTTGCGCTCGCAGGCCTAAGGTCAAGCCC |
| | | GGTTACTTCGTTTGGTAGAACGAGGTAGACGATGC |
| | | CTAGTCTTAAGGTGGCCCATGTTAACCAACAGGGC |
| | | CAGAACATGATTATAGTTCCGTTAGATGCCAACTT |
| | | CGGTTACAAAACCGATGGTGAGCAGTCCGACATCA |
| | | TGTTCGAAATACAGGACGCGGCGCGGTCCGCCGGT |
| | | CTTGCGGGTGCCGTAGTAGCGTTCTGGCAGTCAGG |
| | | TGGACAAACCCGTTTCCGGGGCCCGGCTCCGTGGC |
| | | ACCCATTCCTTCGCAGCCTC |

TABLE 17
Primer and Probe Sequences for Specific
Detection of NLS0662

| | SEQ | |
|---|---|---|
| | ID | |
| Primer/Probe | NO | Sequence* |
| NLS0662_ref1_4871392_forward | 52 | GCGCAAGTCGCAAT |
| | | ACCATTTC |
| NLS0662_ref1_4871392_reverse | 53 | CGTAAATCGGCGGA |
| | | CCTTTGA |
| NLS0662_ref1_4871392_probe | 54 | CGCAGCGGTTATTT |
| | | CGTTG |
| NLS0662_ref1_1266930_forward | 55 | ACGAGCATGCCTCA |
| | | TCCTTATC |
| NLS0662_ref1_1266930_reverse | 56 | CGATTGAGGTGCCC |
| | | GTATGAA |
| NLS0662_ref1_1266930_probe | 57 | TGCCAGTTTGCAGT |
| | | CATTCC |
| NLS0662_ref1_17614_forward | 58 | CCCGGTTACTTCGT |
| | | TTGGTAGAA |
| NLS0662_ref1_17614_reverse | 59 | CGAAGTTGGCATCT |
| | | AACGGAACTA |
| NLS0662_ref1_17614_probe | 60 | TGGCCCATGTTAAC |
| | | CAACAG |

*Bold and underlined letters represent the position of an LNA base

### Use of Primer/Probes for Detection of NLS0807 on Treated Plant Materials

Detection of NLS0807 from In-Furrow Treated Corn Roots

At planting, corn seeds in soil were drenched with NLS0807 and control strains from frozen glycerol stock to simulate in-furrow treatment. To obtain a final concentration of 10⁷ CFU/seed, 100 ul of each strain at 10⁸ CFU/ml is inoculated onto each seed placed in the dibble holes in soil. A 1/10 dilution series is made for lower concentration targets. For control treatment, 100 ul Milli-Q water is applied to each corn seed placed in the dibble holes in soil. Pots containing treated seeds are placed in a growth chamber for approximately two weeks and watered with unfertilized RO water every 1-2 days to keep soil moist. After 2 weeks of growth, roots of about 9 plants per replicate sample were harvested into sterile tubes. Each treatment had at least 2 replicate samples in each experiment, and each experiment was conducted at least 3 times.

DNA from bacteria on the harvested corn roots is isolated as follows. Individual roots are submerged in 20 mL of phosphate-buffered saline (PBS) (137 mM NaCl, 10 mM Phosphate, 2.7 mM KCl, and a pH of 7.4) in 50 mL conical tubes. Tubes are vortexed for 10 minutes, and then sonicated for 10 minutes. Root tissue is removed, and the remaining supernatant from multiple roots of the same sample are combined and centrifuged at 7500×g for 10 minutes. This process is repeated until there is one tube for each sample. The moist soil pellet is vortexed until it evenly coats the tube wall. Tubes are placed into a laminar flow hood with caps removed and open ends of the tubes facing the air blowers. Once dry, samples are stored at room temperature. 250 mg dried soil is used as input for DNA extraction using Qiagen DNeasy PowerSoil HTP 96 kit (Cat #12955-4) using manufacturer protocols.

Primers and probes for NLS0807 disclosed in Table 12 above are used in qPCR reactions to detect the presence of NLS0807 specific fragments provided in Table 11. Each 10 ul qPCR reaction contains 5 ul of Quantabio PerfeCTa qPCR ToughMix 2× Mastermix, Low ROX from VWR, 0.5 ul of 10 uM forward primer, 0.5 ul of 10 uM reverse primer, 1 ul of 2.5 uM probe, 1 ul nuclease free water and 2 ul of DNA template. Approximately 1 ng of DNA template is used per reaction. The reaction is conducted in a ThermoFisher QuantStudio™ 6 Flex Real-Time PCR System with the following program: 95° C. for 3 min, then 40 cycles of 95° C. for 15 sec and 60° C. for 1 min. The analysis software on the PCR instrument calculates a threshold and Ct value for each sample. Each sample is run in triplicate on the same qPCR plate. A positive result is indicated where the delta Ct between positive and negative controls is at least 5.

### Use of Primer/Probes for Detection of Variants of Additional Table 1 Methylobacterium Isolates

Variants of Methylobacterium isolates listed in Table 1 are identified by the presence of DNA fragments as described above. Unique fragments for use in such methods are provided in Table 18.

TABLE 18

| | | SEQ | |
|---|---|---|---|
| | | ID | |
| Strain | Fragment | NO | Sequence |
| NLS0020 | ref3_25009 | 61 | GCCCTTCTGTCAGGCGATATTGTATAATGGCGTTGCCC |
| | | | CAATAGAAGCAGCCATTCGTGCGAGGGCAGCAGCGA |
| | | | CGCTAGGTCGAAAGAGCATCCTAATCTCGATCAAGAT |
| | | | GCGACTGAGATTTCTGATGAAAATATCTAGACACAAG |
| | | | CAAAGCTGGTGAAATTACAACGATCATGGCGACAATT |
| | | | GCGGCCAATTCGGCCGGAACTTGAAGGAACATAAAA |
| | | | ATGAATATTACAAATATACCGCAAAGCATGTAGAGTT |
| | | | GCTACACCAAGGGTCGGGACGTCCAAAAAAACTCACT |
| | | | GAGGA |
| NLS0020 | ref3_25219 | 62 | GGAACATAAAAATGAATATTACAAATATACCGCAAA |
| | | | GCATGTAGAGTTGCTACACCAAGGGTCGGGACGTCCA |
| | | | AAAAAACTCACTGAGGAAGTCGACTGGAAGCACGAG |
| | | | GCGCCCCCCCCAGGAGCGGGGCGACCGGCAAGGGGG |
| | | | CCCGCAATTGTCGCCATGATCGACCAGCTTAGGTAGG |
| | | | ATCCTCTTTCGACCTAACGAATGGCTGCTTCTATTGGG |
| | | | GCAACGCCATTATACAATATCGCCTGACCATCTGGAA |
| | | | CGCGGCCCGGTCCACCGGCAGGTTGGCGACGACAGC |
| | | | GTCGGAG |
| NLS0020 | ref1_4361220 | 63 | CGGCGTCGACCAGCCGGGCGAACTGCTTGGGCATGCT |
| | | | CTCCCGCGACGCCGGCCACAGCCGCGTCCCCGTCCCT |
| | | | CCGCACAGGATCATCGGGTGGATTTGAAAGGCAAAA |
| | | | CGGGACATCAGGATAGGCCGCTCAGGCGTTGGCGCTG |
| | | | AGGCGCTTGATGTCGGCGTCGACCATCTCGGTGATCA |
| | | | GCGCCTCGAGGCTGGTCTCGGCCTCCCAGCCGAAGGT |
| | | | CGCCTTGGCCTTGGCGGGGTTGCCCAGCAGCACCTCG |
| | | | ACCTCTGCCGGCCGGAACAGCGCCGGGTCGACGATCA |
| | | | GGTGG |
| NLS0020 | ref1_4602420 | 64 | CTGGACATGCGCCCACCCCGGCCAAGTCCGACCGCAC |
| | | | CGGCAACCGCTCCTGTAGTCGTCGTCATCGTTCTCACC |
| | | | CCTGAGGCGGAGACCGTCCGCTAACGGGGTGTCTCAA |
| | | | GCAACCGTGGGGCGGAGGAACACGCACGTAGTCGCG |
| | | | TTTCAAGGTTCGCACGAACGCCTCGGCCATGCCGTTG |
| | | | CTCTGCGGGCTCTCCAGCGGCGTCGTTTTTGGCACCA |
| | | | AACCAAGGTCGCGGGCGAAGCGGCGCGTGTCGCGGG |
| | | | GACTGTCAGGAATTTCGTGTGGGGGCGGCCATAGTGG |
| | | | ATCCG |
| NLS0089 | ref1_194299 | 65 | GGAAATCGGCTTCAAGTACGACGTCACGCCGGCCATG |
| | | | CAGGTCACGGGTGCACTGTTCAATCTCGAGCGCGACA |
| | | | ACCAGCCGTTCCCCTCGAACGTGGAGTCCGGCCTCGT |
| | | | CCTTGGCGCAGGTCAGACACGCACCCAGGGCGCGGA |
| | | | AATCGGCCTGGCCGGCTATCTAACCGATTGGTGGCAG |
| | | | GTCTTTGGCGGCTACGCTTATACCGAGGCACGCGTAC |
| | | | TCTCGCCACTGGAAGACGATGGAGACGTGATCGCAGC |
| | | | AGGTAATCTCGTCGGCAACGTTCCGCTAAATACTTTC |
| | | | AGTCT |
| NLS0089 | ref1_194305 | 66 | CGGCCTGGCCGGCTATCTAACCGATTGGTGGCAGGTC |
| | | | TTTGGCGGCTACGCTTATACCGAGGCACGCGTACTCT |
| | | | CGCCACTGGAAGACGATGGAGACGTGATCGCAGCAG |
| | | | GTAATCTCGTCGGCAACGTTCCGCTAAATACTTTCAGT |
| | | | CTGTTCAACAAGTTCGATATCAACGAGAATTTCTCCG |
| | | | TTGCTCTGGGCTATTACTATCAGGATGCCAGCTTTGCC |
| | | | TCCTCAGACAATGCAGTGCGTTTGCCAAGTTATTCGC |
| | | | GGTTCGATGGCGGGTTGTTCTATCGATTCGACGAGTT |
| | | | GAC |
| NLS0089 | ref1_194310 | 67 | ACGTTCCGCTAAATACTTTCAGTCTGTTCAACAAGTTC |
| | | | GATATCAACGAGAATTTCTCCGTTGCTCTGGGCTATTA |
| | | | CTATCAGGATGCCAGCTTTGCCTCCTCAGACAATGCA |
| | | | GTGCGTTTGCCAAGTTATTCGCGGTTCGATGGCGGGTT |
| | | | GTTCTATCGATTCGACGAGTTGACACGCGTTCAGCTT |
| | | | AGCGTCGAGAACATTTTCGACAGGCGTTACATCATCA |
| | | | ACTCCAACAACAACAACAACCTCACGCCTGGCGCGCC |
| | | | GAGAACAGTCCGCGTGCAATTGATCGCTCGGTTCTAA |
| | | | A |
| NLS0042 | ref1_86157 | 68 | AGCCCACAAGCCTGATGCACTTAACTACATCCTCTAA |
| | | | TGTCGCGCCAATTTGCTTGGCGGCAGGGGATGTTGTA |
| | | | TCGTCATAGGCTTGTCTAACCGGAACTTGTTTGCCAAT |
| | | | CTCTTTGGCGATCGCAACCGCCATCTCGTGTTCGTCAA |
| | | | CCATGTGCGCGTTCCTCTAATTGCACTCATGGTGCCAC |
| | | | GTGCACCTCCGATCGTCTCGTGTCTAGAATGAAGGTG |
| | | | GGAACAACCTTACACAGGCTTTCGCGACGCGCGAATT |
| | | | TCTGGTTTCTCCGCCTCGGATGTGGGTTTGAGCGCTTC |
| NLS0042 | ref1_142469 | 69 | CTTTTCATTTGTCATGATCTCGACCAAGGTATTCACGG |
| | | | CAAGCTCGGTCTGTTGCTTAGCAAGTGCCTGAACTTC |
| | | | GCGAACGATCGGCTCTCGACCCTTCGGGTTCGAGACC |
| | | | TGTCCCTTTTGAAAACCACGTGCCCTACACTTTTCGGG |
| | | | ATCAAGGTGCGGGTTGGCTTTGGTCAAAATTCTCTGG |
| | | | CGTCCCATTACACGCCCTCCGCATCATCGTTCCCGCGA |
| | | | ACGATCTGACCCCCGACTTCCGCGAGGAAGCGTGTGG |
| | | | CGTGATCCTCGAAGCGGAATGCCACCTCGAACTGTTC |
| | | | C |
| NLS0042 | ref1_142321 | 70 | CAGCAGCAAGCAGATCGTTGAAAACCGCTTGAACCGC |
| | | | ATCTTGATCGGGACCGGAACCAATCAGGTCATCTAGG |
| | | | TAAACCGAGACGTAAACTCGTTTGCGCTCGGCATCTT |
| | | | TCAGAACGTCCGTGATGCCAGACCGCATTAGTACCAT |
| | | | CGTCGCCAAGGCGGGCGACTGAACGAAGCCGATCGG |
| | | | CAGAGAGTAACGGGGACCGCCCCTAATCGGGTTGCG |
| | | | AACGCAAGACCACTTAGCAAAGGTTCGAGCACGGCC |
| | | | GAACTTCGCATGGTGGAGAGCCGCGGCAACACGGTTC |
| | | | CGTGATA |
| NLS0064 | ref1_153668 | 71 | TAGACATTCCAACAAACCGGCAAGAGGCTCGTCCTCA |
| | | | CTCGAGGATTTGTTGGGACTTGCATGATGTCGAAGCG |
| | | | GAGCCGTTATGACCTGGGTGCGATCATGCGCCGAGCA |
| | | | TGGGAGATGGCTCGGGAGGCGGCATTCGCGGTTGGCG |
| | | | AGCGGGCACGGACTCACCTTGCTGCCGCGATGCGCAG |
| | | | CGCGTGGGCCGAAGCCAAGTTGGCACTCGCGCCCACG |
| | | | AAGACGGAGCAGGATCGTCTCTCTCCGAGCGACATGA |
| | | | TCGGACATGAGGACGCCTACCAAGGCCGGGTTCTAAA |
| | | | ATAT |
| NLS0064 | ref1_3842117 | 72 | AAGATGGATACGACAAGCGCGATTACATTATTTGCGA |
| | | | AATAGATGGACAAATAAAAGACAAAGGACTGATGTA |
| | | | TTTCCTTAAATCTGGACAAGTTGACCTCTTTCACATAG |
| | | | AAGTCACCACTCCCTTTGGGACAATTTGGTGTCACGA |
| | | | AAACATAGAGGCCGAACTTCTTAGCTGAATTATCGCG |
| | | | CTCCGGGTTCTTATGCGGCTGAGTGAAGCGCGGGACA |
| | | | GCTTGCGAGCAGGGCCGCCAATGGCAGCCGGGATGA |
| | | | CACAATGCTCGGTCTCCCGACGCTTCTTCAATCGGGA |
| | | | GCGCT |
| NLS0064 | ref1_3842278 | 73 | AGCTGAATTATCGCGCTCCGGGTTCTTATGCGGCTGA |
| | | | GTGAAGCGCGGGACAGCTTGCGAGCAGGGCCGCCAA |
| | | | TGGCAGCCGGGATGACACAATGCTCGGTCTCCCGACG |
| | | | CTTCTTCAATCGGGAGCGCTTCGCAGCCCGGGGCGGC |
| | | | GCGCTCATGCGTCACGACCTGGGCCCTGCGCACCTTC |
| | | | GCGGCCCCGCCGTCCCGGCAGATCCCTGATGCCCCAA |
| | | | GTGGGCGGCCACTCCATCAAAGAACCCCGGCCTGTGG |
| | | | CAGATCTCGTAGGCATACCGAGGTTCCGCAGTGCCCC |
| | | | CACC |
| NLS0610 | ref1_2810264 | 74 | ACCGAAGGCGTCCCCGGACACGAAGGCCTGAAACAC |
| | | | CATATCTGTGGCGATCAGGCCGACGTGGTCGCGGACT |
| | | | TCAACTGGCAGAGAATGCCAGGCCGCTTCGATTTCAG |
| | | | ATGATACTGGTACGGACATAGGAGCGGCTTAGCTTTC |
| | | | TCAGTGCAAATGTGATTGATTCCGGCTCAAAAATGAT |
| | | | CTTGATCGGACGAGACGTTTTCAATCCATGTCGTGTTG |
| | | | CCATCGCCGATCGGTGCGTCAAGAGACAGATGGCGCC |
| | | | GACCGTAGATACGCGTTCGGGTTGCCCGCACCGCTTC |
| | | | TCCA |
| NLS0610 | ref1_322980 | 75 | GGAGGTGTGATCTGATGATGTGCTGGATGAAATT |
| | | | GGCGGTCGAGCACTTGTTCAGCTTGGCCAGCTCG |
| | | | ACGAGATCGGCGTGATGCTCGGCGTCGATCAGGA |
| | | | TGTTCAGCGAGACCGGACGTACGCAGGACTTGGT |
| | | | ATTAGCGCCGTTGCGCATCAGCTTGCAGCCTTGC |
| | | | TCTGCTTCTCAGCGTGCCGCGTCAGGATGACCCT |
| | | | GATGTAGCTGTTGAGGTTGATGCCGTAATAGCCT |
| | | | GCGGACTCTGTGAGATCCCGGCGAAGATCGTCGG |
| | | | CGAGGGTCAGGCGGATGGTGCTGGTCGG |
| NLS0610 | ref1_2785241 | 76 | AAGTAACCGCTCAACATGATCTTCAGCATGTTGT |
| | | | CCAACAGCAGGAGAATACATGTAATTCACCATGA |
| | | | CCGGCAAGCTGCGACTGGCCATTGCTTCCACCGC |
| | | | TTGAATGTAGCGATCGAATTTCGCAAAATCAGGG |
| | | | TGGAATGAAAATATCGAACCAAACTGCGAGCCTT |
| | | | GAATCCGTTCTGCAAAATTATCGAAAAATTTTCT |
| | | | TGGCCGACTGCCGTTCGAAAACATTCTTACGTTT |
| | | | ACATGCGGCCCGCCTGAAACAAGACAGTCTACCA |
| | | | GCTCTGGGAAATGGGGGTGAAGGGTCGG |

### Example 5. Analysis of Effects of Methylobacterium Strains on Nutrient Content of Soybean Vegetative Tissues and on Oil, Protein and Nutrient Content of Seeds of Canola, Corn, Soybean and Wheat

Soybean seeds treated as described in Example 1 were grown in multiple field locations in the Midwestern United States in the summer of 2019 in parallel with untreated control soybean plants. Seeds from Canola and wheat were similarly treated and tested. For analysis of field grown corn plants, Methylobacterium strains were applied in-furrow at planting. Strains and strain combinations evaluated are shown in Table 19 below.

TABLE 19

| Crop | Methylobacterium strain(s) |
|---|---|
| Canola | NLS0017 |
| Canola | NLS0020 |
| Canola | NLS0064 |
| Canola | NLS0610 |
| Canola | NLS0017 + NLS0064 |
| Canola | NLS0064 + NLS0066 |
| Canola | NLS0017 + NLS0109 |
| Corn | NLS0042 |
| Corn | NLS0807 |
| Corn | NLS0020 + NLS0042 |
| Corn | NLS0042 + NLS0807 |
| Corn | NLS0648 |
| Corn | NLS0662 |
| Corn | NLS0017 + NLS662 |
| Soybean (+Rhizobia treatment) | NLS0064 |
| Soybean (+Rhizobia treatment) | NLS0610 |
| Soybean (+Rhizobia treatment) | NLS0934 |
| Soybean (+Rhizobia treatment) | NLS0017 + NLS0109 |
| Soybean | NLS0017 |
| Soybean | NLS0064 |
| Soybean | NLS0089 |
| Soybean | NLS0109 |
| Soybean | NLS0020 |
| Soybean | NLS0648 |
| Soybean | NLS0017 + NLS0109 |
| Soybean | NLS0807 |
| Wheat | NLS0017 |
| Wheat | NLS0021 |
| Wheat | NLS0044 |
| Wheat | NLS0064 |
| Wheat | NLS0089 |
| Wheat | NLS0109 |

Preliminary analysis of soybean vegetative tissue indicates increased micronutrients were obtained by treatment with Methylobacterium strains, including increased boron in R1 stage vegetative tissue in soybean plants grown from ISO003 and ISO018-treated seeds, and increased iron in V6 stage vegetative tissue in soybean plants grown from ISO002-treated seeds.

### REFERENCES

Bradford, M. M. A rapid and sensitive method for the quantitation of microgram quantities of protein utilizing the principle of protein-dye binding. Anal. Biochem. 1976, 72, 248-254. Dumas, Ann. Chim. Phys. 1831, T47, 198-213.
Green, P. N. 2005. Methylobacterium. In Brenner, D. J., N. R. Krieg, and J. T. Staley (eds.). “Bergey's Manual of Systematic Bacteriology. Volume two, The Proteobacteria. Part C, The alpha-, beta-, delta-, and epsilonproteobacteria.” Second edition. Springer, New York. Pages 567-571.
Green, P. N. 2006. Methylobacterium. In Dworkin, M., S. Falkow, E. Rosenberg, K.-H. Schleifer, and E. Stackebrandt (eds.). “The Prokaryotes. A Handbook on the Biology of Bacteria. Volume 5. Proteobacteria: Alpha and Beta Subclasses.” Third edition. Springer, New York. Pages 257-265.
Green, P. N. and Ardley, J. K. 2018. Review of the genus Methylobacterium and closely related organisms: a proposal that some Methylobacterium species be reclassified into a new genus, Methylorubrum gen. nov. Int J Syst Evol Microbiol. 2018 September; 68(9):2727-2748. doi: 10.1099/ijsem.0.002856.
Hartree, E. F. Determination of protein: a modification of the Lowry method that gives a linear photometric response. Anal. Biochem. 1972, 48, 422-427.
Jokić, S., Nagy, B, Zeković, Z., Vidovid, S., Bilić, M., Velid, D., Simándi, B. 2012 Effects of supercritical CO₂ extraction parameters on soybean oil yield. Food and Bioproducts Processing 90(4): 4, 693-699.
Konstantinidis K. T., Ramette A., Tiedje J. M., (2006). The bacterial species definition in the genomic era., Philos Trans R Soc Lond B Biol Sci 361, 1929-1940.
Kjeldahl, J. Anal. Chem. 1883, 22, 366-382.
Lidstrom, M. E. 2006. Aerobic methylotrophic prokaryotes. In Dworkin, M., S. Falkow, E.
Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.
MoThre, H. K., Dalheim, L., Edvinsen, G. K., Elvevoll, E. O., and Jensen, I. J. (2018) Protein Determination-Method Matters. Foods 7(1), 5; doi:10.3390/foods7010005.
Sy, A., Giraud, E., Jourand, P., Garcia, N., Willems, A., De Lajudie, P., Prin, Y., Neyra, M., Gillis, M., Boivin-Masson, C., and Dreyfus, B. 2001. Methylotrophic Methylobacterium Bacteria Nodulate and Fix Nitrogen in Symbiosis with Legumes. Jour. Bacteriol. 183(1):214-220.
Thiex, N.J., Anderson, S., and Gildemeister, B. 2003: Journal Of AOAC International VOL. 86, NO. 2, 2003.

The breadth and scope of the present disclosure should not be limited by any of the above-described embodiments, but should be defined only in accordance with the following claims and their equivalents.

What is claimed is:

1. A method of producing a food or feed with increased mineral nutrient, crude fat, oil, and/or protein content comprising incorporating into the food or feed a processed or unprocessed food ingredient obtained from a cultivated plant or plants grown from Methylobacterium-treated seeds, plants or plant parts, wherein the Methylobacterium is ISO20 (NRRL B-67743), ISO19 (NRRL B-67742), ISO18 (NRRL B-67741), ISO26 (NRRL B-67892), or variant thereof, thereby obtaining a food or feed with increased mineral nutrient, vitamin, oil and/or protein content.

2. The method of claim 1, wherein the mineral nutrient is selected from nitrogen, phosphorus, potassium, iron, boron, magnesium and manganese.

3. The method of claim 1, wherein said variant of ISO20 (NRRL B-67743) comprises a sequence selected from the group consisting of SEQ ID NO: 25, 26, and 27; wherein said variant of ISO18 (NRRL B-67741) comprises a sequence selected from the group consisting of SEQ ID NO: 37, 38, and 39; wherein said variant of ISO19 (NRRL B-67742) comprises a sequence selected from the group consisting of SEQ ID NO: 49, 50, and 51, or wherein said variant of ISO26 (NRRL B-67892) comprises a sequence selected from the group consisting of SEQ ID NO: 74, 75, and 76.

4. The method of claim 1, wherein said plant or plants are a soybean, Brassica sp., corn, sunflower, cotton, flax, alfalfa, rice, rye, wheat, barley, oat, sorghum, millet, safflower, or peanut plant or plants.

5. A plant or plant part having increased mineral nutrient, crude fat, seed oil, and/or protein content, wherein said plant, seed or plant part is harvested from a cultivated plant grown from a Methylobacterium-treated seed, plant or plant part, wherein said Methylobacterium provides for increased mineral nutrient, crude fat, seed oil, and/or protein content, and wherein the Methylobacterium strain is ISO20 (NRRL B-67743), ISO19 (NRRL B-67742), ISO18 (NRRL B-67741), ISO26 (NRRL B-67892), or variant thereof.

6. The plant part according to claim 5, wherein said plant part is a seed, leaf, stem, flower, root, fruit, or tuber.

7. The plant part according to claim 5, wherein said plant part is a seed having increased seed oil, protein and/or mineral nutrient content.

8. The plant part of claim 5, wherein said variant of ISO20 (NRRL B-67743) comprises a sequence selected from the group consisting of SEQ ID NO: 25, 26, and 27; wherein said variant of ISO18 (NRRL B-67741) comprises a sequence selected from the group consisting of SEQ ID NO: 37, 38, and 39; wherein said variant of ISO19 (NRRL B-67742) comprises a sequence selected from the group consisting of SEQ ID NO: 49, 50, and 51; or wherein said variant of ISO26 (NRRL B-67892) comprises a sequence selected from the group consisting of SEQ ID NO: 74, 75, and 76.

9. The plant part of claim 7, wherein the mineral nutrient is selected from the group consisting of nitrogen, phosphorus, potassium, iron, boron, magnesium and manganese.

10. The plant part of claim 7, wherein protein content is increased by at least about 0.5%, 1%, or 2% per gram dry or wet weight of the seed in comparison to protein content of a control seed.

11. The plant part of claim 5, wherein the plant part is a soybean, Brassica sp., corn, sunflower, cotton, flax, alfalfa, rice, rye, wheat, barley, oat, sorghum, millet, safflower, or peanut plant part.
